(19) United States
(12) Patent Application Publication
(10) Pub. No.: US 2011/0165680 A1
(43) Pub. Date: Jul. 7, 2011

(54) CLEAN GENOME BACTOFECTION

(75) Inventors: Frederick R. Blattner, Madison, WI (US); Val Burland, Cross plains, WI (US); Charles Landy, Fitchburg, WI (US); Nian-Qing Shi, Middleton, WI (US); Buffy Stahl, Oregon, WI (US)

(73) Assignee: Scarab Genomics, LLC, Madison, WI (US)

(21) Appl. No.: 13/063,450

(22) PCT Filed: Sep. 14, 2009
(86) PCT No.: PCT/US2009/056829

§ 371 Date: Mar. 10, 2011

Related U.S. Application Data
(60) Provisional application No. 61/096,649, filed on Sep. 12, 2008.

(51) Int. Cl. C12N 15/85; C12N 1/21; C07H 21/04; C12N 15/63; C07K 14/00

(52) U.S. Cl. 435/454; 435/252.3; 536/23.1; 435/320.1; 530/300

(57) ABSTRACT

Methods for introducing and expressing genes in animal cells are provided comprising infecting the animal cells with live invasive reduced-genome bacteria comprising a eukaryotic expression cassette comprising said gene. Also provided are methods for producing a pluripotent stem (iPS) cell from a mammalian somatic cell comprising infecting the somatic cell with live invasive reduced-genome bacteria comprising one or more eukaryotic expression cassettes comprising at least a gene encoding the transcription factor Oct3/4 and a gene encoding a member of the SRY-related HMG-box (Sox) transcription factor family.

### CROSS-REFERENCE TO RELATED APPLICATIONS

This application claims the benefit of U.S. Provisional Application No. 61/096,649, filed Sep. 12, 2008, the contents of which are incorporated herein by reference.

### FIELD OF THE INVENTION

The present invention is directed to materials and methods for introducing genes into eukaryotic cells using live invasive bacteria having a clean genome lacking non-essential elements and which comprises an expression cassette capable of expressing a heterologous sequence in an eukaryotic cell and preferably an animal cell.

### BACKGROUND OF THE INVENTION

The use of nucleic acid delivery technology to deliver a nucleic acid (e.g. a functional gene copy or an oligonucleotide) affecting the expression of a target gene in a patient is the basic principle behind gene therapy. In order to achieve the desired result, delivery vectors for nucleic acid transfer are required. The most frequently used vectors include viral vectors derived from adenoviruses, retroviruses, poxviruses and the like. However, naked plasmid DNA, alone or in combination with enhancers of cell-membrane penetration, has been used for short-term applications. Many of these vectors share limitations in production costs, amount of delivered nucleic acid and difficulty of application.

The technique of using live invasive bacteria as a vector for the delivery of nucleic acids into a target organism, tissue, or cell, is known as bactofection. According to this method, a bacterial strain is transformed with a plasmid comprising a eukaryotic expression cassette comprising the nucleic acid of interest. The live, transformed, bacteria are then used to infect target cells, resulting in expression of the eukaryotic expression cassette by the infected cells (and their progeny). U.S. Pat. Nos. 5,877,159; 6,150,170; and 6,682,729 describe the use of certain bacteria to introduce DNA into animal cells and these patents are incorporated by reference herein in their entirety.

Bactofection of a variety of mammalian cells, including phagocytic and nonphagocytic mammalian cells, has been demonstrated. Bactofection efficiency, however, has generally been low. For example, U.S. Pat. No. 5,877,159 discloses bactofection efficiencies of about 20% in HeLa cells, less in macrophages. Pilgrim et al. Gene Therapy 10:2036-2045 (2003), describe an improved bactofection system with a reported efficiency of between 5-20% depending on cell type.

Vaccine development entered a new era with the ability to rationally modify viruses and bacteria using molecular genetics. These modifications include attenuation to a non-virulent phenotype and the inclusion of additional genes encoding disparate immunogens. Two oral live bacterial vaccines are licensed for human use at present: Salmonella enterica serovar Typhi (S. typhi) Ty21a (Berna Biotech Ltd.) and Vibrio cholerae CVD 103-HgR (Berna Biotech Ltd). These live bacterial vaccines have been used for the safe and effective immunization of several million individuals against typhoid fever and cholera, respectively (Dietrich et al. Vaccine 21 (7-8):687-683, 2003).

The ability of bacterial DNA delivery to immunize against viral diseases has also been assessed. For example, infection with herpes simplex virus-2 (HSV-2) can be controlled by strong T-cell responses in the genital mucosa. Oral immunization with S. typhimurium ΔaroA carrying DNA plasmids encoding the HSV-2 glycoproteins D (gD) or B (gB) in mice resulted in strong systemic and mucosal (vaginal) T-cell responses, including vaginal memory T-cells, and conferred protection against a vaginal challenge with HSV. This bacterial delivery demonstrated clear superiority to intramuscular injection of the same plasmid constructs with regard to the level of mucosal T-cells and protection evoked against vaginal challenge with HSV (Flo et al. Vaccine 19(13-14):1772-1782, 2001).

Several studies have shown that bactofection can be used in methods of gene therapy, including delivery of plasmids similar to those used as DNA vaccines. For example, attenuated bacterial vectors can be used as anti-HIV vaccines. The greatest hindrance to the development of an HIV-1 vaccine that induces mucosal immune responses has been the poor immunogenicity of immunogens administered in this compartment. Fouts et al. reported that the Salmonella DNA vaccine vector was capable of delivering a passenger HIV-1 gp120 DNA vaccine to host cells and inducing CD8⁺+T cell responses to gp120. Therefore, it seems that the attenuated bacterial vectors can overcome a problem of poor immunogenicity of immunogens administered to mucosal tissues (Fouts et al. FEMS Immunology and Medical Microbiology 37:129-134 2003).

Attenuated Salmonella and Shigella strains have been used successfully to deliver DNA vaccines in mice against a variety of infectious diseases of both bacterial and viral origin, particularly in models requiring protection by T-cells. For example, S. typhimurium purine auxotrophic strain 22-11 was assessed for the delivery of a DNA vaccine vector encoding the major outer membrane protein of the respiratory pathogen Chlamydia trachomatis. Oral immunization led to partial protection of mice against lung challenge with C. trachomatis, demonstrating that plasmid delivery to the mucosal surface of the gut could elicit immune responses and provide protection at a distant mucosal surface, namely the lung (Brunham et al., Am Heart 138(5 Pt 2): S519-S522 1999).

The use of bacteria-based vaccines need not be limited to infections. For example, cancer may be amendable to such intervention for example by vaccination with self-antigens to induce tumor specific immunity to combat tumor cells. Live bacterial vaccines are well suited to deliver DNA vaccines encoding tumor-specific antigens, as shown in a variety of studies. Furthermore, attenuated Salmonella strains have even been shown to specifically target tumor tissues, which may allow for the selective vaccine delivery into tumor cells (Zheng et al. Oncol. Res. 12(3):127-135, 2000). Studies done so far in the area of tumor DNA vaccine delivery were performed in mice with S. typhimurium ΔaroA as a carrier. The live attenuated bacteria have been successfully applied to the treatment of several tumor types such as melanoma, neuroblastoma and different adenocarcinomas in experimental animals (Dietrich et al., Current Opinion in Molecular Therapeutics 5(1), 10-19, 2003).

Powell et al. in U.S. Pat. No. 5,877,159 (incorporated herein by reference in its entirety) teaches how attenuating mutations can be introduced into pathogenic bacteria using non-specific mutagenesis or recombinant DNA techniques. This attenuation approach can be described as “top down” approach in which a wild-type bacterium is attenuated by removal of one or more genes that are involved in pathogenesis in susceptible hosts. However, even a bacterium in which one or more genes essential for pathogenicity have been deleted, might revert to a pathogenic phenotype in a population of immunized subjects. Such reversion is possible partially because vaccine strains described so far carry a large array of mobile genetic elements such as phage and insertion sequences (IS) that facilitate recombination and consequently, can restore the pathogenic phenotype.

Among the other problems with live attenuated bacterial strains that need to be overcome include the need for very high and/or repeated doses in some cases; plasmids and antibiotic markers used in constructing the strains are still present and could potentially be transferred to other organisms; thirdly, some strains (e.g. Shigella) produce immune responses to bacterial components other than that specifically desired, which can also lead to side-effects. Additionally, there is a need for improved bactofection methods having an increased bactofection efficiency.

### SUMMARY OF THE INVENTION

The present invention is directed to a bacteria having a “clean genome” (alternatively referred to herein as a “reduced genome” or a “multiple deletion strain” [MDS]) for delivering expressible DNA or RNA into an animal cell and methods for doing so. The DNA or RNA may encode or comprise therapeutic or prophylactic agents. This process of delivering such DNA or RNA into cells is referred to herein as “bactofection” and the bacteria used in the methods are referred to as bacterial vectors or bactofection vectors. The clean genome may be produced by deleting selected genes from a native parental strain of a bacterium or may, for example, be entirely synthesized as an assembly of preselected genes selected to provide a bacterium with appropriate growth and metabolic properties to serve as a delivery vehicle for the heterologous expressible sequences.

In one embodiment, the clean genome bacteria used in the practice of the present invention have a genome that is preferably genetically engineered to be at least two percent (2%) and up to twenty percent (20%) (including any integer therebetween) smaller (1%) than the genome of a native parent strain. Preferably, the genome is at least seven percent (7%) smaller than the genome of a native parent strain including any integer therebetween smaller than the genome of the native parent. More preferably, the genome is eight percent (8%) to fourteen percent (14%) to twenty percent (20%) (including any integer therebetween) or more smaller than the genome of the native parent strain. Alternatively, the genome may be engineered to be less than 20% smaller than the genome of a native parental strain so long as it is designed according to the parameters described herein. For example, a strain may be designed to lack only insertion sequences. The bacterium further comprises expression cassettes which comprise expressible DNA or RNA as described herein.

As described in U.S. patent application Ser. Nos. 10/896,739, 11/275,094, 11/400,711 and U.S. Pat. Nos. 6,989,265 and 7,303,906, the contents of each which is incorporated herein by reference in its entirety, the clean genome bacteria may be engineered to lack, for example, genetic material such as, but not limited to, certain genes unnecessary for growth and metabolism of the bacteria, insertion sequences (transposable elements mobile genetic element), pseudogenes, prophage, undesirable endogenous restriction-modification genes, pathogenicity genes, toxin genes, fimbrial genes, periplasmic protein genes, invasin genes, lipopolysaccharide genes, class III secretion systems, phage virulence determinants, phage receptors, pathogenicity islands, RHS elements, sequences of unknown function and sequences not found in common between two strains of the same native parental species of bacterium. Other DNA sequences that are not required for cell survival can also be deleted or omitted.

The clean genome bacteria of the present invention also provides a basic genetic framework to which may be added desired genetic elements for expression of useful products as well as genetic control elements which offers an opportunity to fine tune or optimize the expression of the desired product. As is readily apparent from the discussion herein, a clean genome bacterium has fewer than the full complement of genes found in a native parent strain to which it is compared, and with which it shares certain essential genes. However, as discussed above, the word “reduced” should not be construed as a process limitation in that such a bacterial genome may be produced by assembling selected genes de novo into a synethetic genome using the design parameters described and only incorporated herein.

In one embodiment, the present invention is directed to methods of bactofection using the clean genome bacteria. Preferably, bactofection methods of the invention have a bactofection efficiency of greater than 30%, 35%, 40%, 45%, 50%, 55%, 60%, 65%, 70%, 75%, 80%, 85%, 90%, 95%, 96%, 97%, 98% or 99%. More preferably, the bactofection methods of the invention have a bactofection efficiency of greater than 90%, most preferably of greater than 95%.

In a related aspect, the present invention is directed to a method for delivering expressible DNA or RNA into an animal somatic cell in vitro, wherein said DNA or RNA encodes or comprises one or more factors (e.g. transcription factors) which, alone or in combination, are sufficient to induce the generation of pluripotent stem cells (iPS) from said animal somatic cell. The DNA or RNA encoding or comprising one or more factors are preferably of human origin; however, animal orthologs of the factors, such as murine orthologs, are also useful in the invention.

In a related aspect, the present invention is directed to delivering heterologous expressible DNA or RNA encoding or comprising therapeutic or prophylactic agents into an animal cell. The therapeutic or prophylactic agents encoded by the heterologous DNA or RNA may include immunoregulatory agents, antigens, for example, antigens associated with pathogenic organisms or tumors, DNAs, antisense RNAs, catalytic RNAs, proteins, peptides, antibodies, cytokines or other useful therapeutic or prophylactic molecules.

Preferably, the heterologous DNA or RNA comprises a prokaryotic or eukaryotic expression cassette and is preferably capable of replication. Preferably, replication of the expression cassette in the clean genome bacteria and/or animal cells is inducible upon introduction into an animal cell.

The invention is also directed to therapeutic or prophylactic methods in which the bacterial vectors of the present invention and administered to animals, preferably humans, for the purpose of treating or preventing diseases.

In one embodiment, the present invention is directed to the use of a non-pathogenic clean genome strain of E. coli K-12 strain as a vaccine. This strain preferably further comprises a set of invasive or invasion genes, such as the Shigella invasion locus, Salmonella invasion genes, locus the invA gene of Yersinia pseudotuberculosis or genes encoding any other bacterial or parasite invasion system or parts of such systems, so that the reduced genome E. coli acquires an invasive phenotype and can enter animal and preferably human cells. See Isberg et al., Cell 50:769-778, 1987. The clean genome strain may also contain restriction/modification systems (preferably heterologous) to prevent horizontal transition of genetic material. The use of such reduced genome (or clean genome) bacteria obviates problems associated with other live attenuated bacterial vectors such as reversion to pathogenic phenotype, acquisition of genes encoding drug resistance potential immunogenicity of the bacterial vector and requirements for repeated immunization doses.

### DESCRIPTION OF THE DRAWINGS

FIG. 1. pBAC3, Map of the copy number amplifiable vector.

FIG. 2. Amplification of the 30 kb invasion locus of Shigella.

FIG. 3. Expression of LacZ in Eukaryotic cells.

FIG. 4. Bactofection of lacZ. Shigella flexneri 2a vaccine strains CVD 1203 (22) and CVD 1208 (32) were transformed with the gWIZ-LacZ expression plasmids that contain an intron in the LacZ coding region. The expression-negative clone served as a control for these experiments. The transformed Shigella strains were checked for Congo red staining and IpaB expression to confirm the presence of the virulence plasmid bearing the invasion locus. Colonies positive for both were selected for bactofection experiments. HeLa cells (5×10⁴ per well) were incubated for 2 h with a late log phase cultures of the appropriate bacteria at a MOI of 5:1. After 2 h the cells were rinsed 5× with media containing 100 ug/ml Gentamicin and then incubated overnight in the same medium. At 21 h the cells were fixed for 5 min and then stained with X-gal as per manufacturers protocols to visualize β-galactosidase expression.

FIG. 5. Immunogenicity LacZ-intron in a human primary in vitro response system.

FIG. 6. Alignment of Stx1A and Stx2A.

FIG. 7. Adherence and Invasiveness of MDS43+/−pBAC3-invA.

FIG. 8. pYinv4, Map of the copy number amplifiable vector.

FIG. 9. High Efficiency Bactofection. Reduced genome strain. MDS42(recA)(ryhb)(trfA⁺) comprising a β-galactosidase expression plasmid with an intron within the lacZ gene, was used to infect HeLa cells. Panel A demonstrates that a bactofection efficiency of 0% is observed (no blue HeLa cells following staining with X-gal) if high copy number of the expression plasmid is not induced prior to infection. Panel B demonstrates that when high copy number of the expression plasmid is induced, the bactofection efficiency improves to about 37%. Panels C and D demonstrate that when the bacteria is frozen in 15% glycerol following induction of the expression plasmid to high copy number, the bactofection efficiency improves to about 99%.

FIG. 10 shows the nucleotide sequence (SEQ ID NO: 5) of a vaccine gene encoding immunogenic Stx2 epitopes (StxA-1 (SEQ ID NO: 1), StxA-4 (SEQ ID NO: 2), StxA-6 (SEQ ID NO: 3) and StxB-1 (SEQ ID NO: 4)) combined end-to-end, in frame, though not in the order in which they occur in the native Stx2 genes. The nucleotide sequence is codon-optimized for E. coli expression.

### DETAILED DESCRIPTION OF THE INVENTION

There remains a need for improved bacterial vectors which have, inter alia, a stable, reduced genome lacking, for example, insertion sequences, and other non-essential genes and which are preferably engineered to protect against horizontal transfer of genetic information that may, for example, destabilize the genome or confer antibiotic resistance to the bacteria and which are capable of invading eukaryotic cells, preferably animal cells including human cells and delivering to the cells expressible nucleic acid including, without limitation, nucleic acid encoding therapeutic and/or prophylactic agents and nucleic acid encoding or comprising one or more factors which, alone or in combination, are sufficient to induce the generation of pluripotent stem cells (iPS) from animal somatic cells. Exemplary embodiments of the present invention described herein include clean genome E. coli based bacterial vectors and methods for bactofection using the clean genome E. coli based bacterial vectors with improved bactofection efficiency.

### I. Clean Genome Bacteria

It is assumed that at least part of the DNA sequence of the target bacterial strain, bacteriophage genome, or native plasmid is available. Preferably, the entire sequence is available. Such complete or partial sequences are readily available in the GenBank database. The full genomic sequences of several strains of E. coli have been published (for example, Blattner et al, Science, 277:1453-74, 1997 K-12 Strain MG1655; See also GenBank Accession No. U00096; Perna et al, Nature, 409, 529-533, 2001; Hayashi et al, DNA Res., 8, 11-22, 2001, and Welch et al., Proc. Natl. Acad. Sci., USA (2002) 99 (26) 17020-17024 and GenBank Accession No. AE014075, all of which are incorporated herein by reference in their entirety), as is the sequence of several other commonly used laboratory bacteria where sequences are found in GenBank.

One type of E. coli DNA element, that can be deleted is the IS elements (or transposable elements). IS elements are not important for bacteria survival and growth in a cultured environment and are known to interfere with genome and plasmid stability. Thus, the IS elements can be deleted in generating a bacterium with a smaller genome.

Another type of E. coli DNA element that can be deleted include the Rhs elements. All Rhs elements share a 3.7 Kb Rhs core, which is a large homologous repeated region (there are 5 copies in E. coli K-12) that provides a means for genome rearrangement via homologous recombination. The Rhs elements are accessory elements which largely evolved in some other background and spread to E. coli by horizontal exchange after divergence of E. coli as a species.

Still another type of region in the E. coli genome that can be deleted is the non-transcribed regions because they are less likely to be important for cell survival and proliferation.

Prophages, pseudogenes, toxin genes, pathogenicity genes, periplasmic protein genes, membrane protein genes are also among the genes that may be deleted, based on the gene selection paradigm discussed herein. After the sequence of E. coli K-12 (see Blattner, et al., supra), was compared to the sequence of its close relative 0157:H7 (See Perna et al., supra) and it was discussed that 483/4288 or 11.3% (K-12) and 1387/5416 or 26% (O157:H7) of the protein encoding genes were located on strain specific islands of from one to about 85 kb inserted randomly into a relatively conserved backbone.

Among other genes that may be deleted are genes that encode bacteriophage receptors including, for example, tonA (fhuA) and/or its complete operon fhuABC which encodes the receptor for the lytic phage T1.

Particular design parameters and methods for producing the reduced (or clean) genome strains of the present invention are described in U.S. patent application Ser. Nos. 10/057,582; 10/655,914 and PCT/US03/01800 which are incorporated herein by reference in their entirety. As is readily apparent, the engineering aspect of the present invention is not limited to reducing a genome per se but also, includes a process of engineering from the bottom-up. That is, a minimal or reduced genome may be constructed by assembling essential genes into an artificial genome which can be used to replace an existing genome in a bacterium or to create a bacterium de novo. Preferably the clean genome bacterium have a genome that is at least two percent (2%), preferably over five percent (5%), more preferably over seven percent (7%) to eight percent (8%) to fourteen percent (14%) to eighteen percent (18%) to twenty percent (20%), to forty percent (40%) to sixty percent (60%) smaller than the genome of its native parental strain. The term “native parental strain” means a bacterial strain (or other organism) found in a natural or native environment as commonly understood by the scientific community and on whose genome a series of deletions can be made to generate a bacterial strain with a smaller genome. Native parent strain also refers to a strain against which the engineered strain is compared and wherein the engineered strain has less than the full complement of the native parent strain. The percentage by which a genome has become smaller after a series of deletions is calculated by dividing “the total number of base pairs deleted after all of the deletions” by “the total number of base pairs in the genome before all of the deletions” and then multiplying by 100. Similarly, the percentage by which the genome is smaller than the native parent strain is calculated by dividing the total number of nucleotides in the strain with the smaller genome (regardless of the process by which it was produced) by the total number of nucleotides in a native parent strain and then multiplied by 100.

Preferably a bacterium according to the present invention comprises a reduced genome bacterium in which about 5% to about 10% of its protein coding genes are deleted. Preferably about 10% to 20% of the protein coding genes are deleted. In another embodiment of the invention, about 30% to about 40% to about 60% of the protein encoding genes are deleted. In addition to deletion of protein encoding genes other non-essential DNA sequences discussed above are also deleted.

Alternatively, the clean genome bacteria of the present invention have a genome less than 2% smaller than the genome of the native parental strain from which certain classes of genetic elements are lacking, (i.e., lacking any IS sequence or certain other native genetic elements).

Generally speaking, the types of genes, and other DNA sequences, that can be deleted are those the deletion of which does not adversely affect the rate of survival and proliferation of the bacteria under specific growth conditions. Whether a level of adverse effect is acceptable depends on a specific application. For example, a 30% reduction in proliferation rate may be acceptable for one application but not another. In addition, adverse effect of deleting a DNA sequence from the genome may be reduced by measures such as changing growth conditions. Such measures may turn an unacceptable adverse effect to an acceptable one. Preferably, the proliferation rate is approximately the same as the parental strain. However, proliferation rates ranging from about 5%, 10%, 15%, 20%, 30%, 40% to about 50% lower than that of the parental strain are within the scope of the invention. More particularly, preferred doubling times of bacteria of the present invention may range from about thirty minutes to about four hours.

The choice of genome segments to be deleted drawn on insights into the genome structure following the sequencing of several whole E. coli genomes. One of the preferred embodiments of the instant invention discloses islands acquired by horizontal genetic transfer. This information was obtained by comparing the genome of the ‘benign’ K-12 strain with several pathogenic strains. Some islands contain non-essential DNA that is undesirable for a vaccine strain. A stable and ‘cleaned-up’ bacterium would be a significant advantage. A minimal strain might consist of the backbone (regions in common with other E. coli), having about 3700 genes. This still includes considerable redundant functions and would constitute a robust set of genes that has stood the test of evolution.

E. coli is used herein as an example to illustrate the genes and other DNA sequences or elements that are candidates for deletion in order to generate a bacterium that can serve as an efficient bactofection vector. The general principles illustrated and the types of genes and other DNA sequences identified as candidates for deletion are applicable to other bacteria species or strains. It is understood that genes and other DNA sequences identified below as deletion candidates are only examples. Many other E. coli genes and other DNA sequences not identified may also be deleted without affecting cell survival and proliferation to an unacceptable level and such genes are readily identified using methods described herein.

Preferred embodiments of the instant invention include rationally designed modifications of the E. coli genome such as removal of phage receptors, removal of intracellular, periplasmic and membrane proteinases, as well as all recombinogenic or potentially mobile sequences and horizontally transferred segments. The techniques involve various ways of forcing homologous recombination in vivo, such that even large 100 kb) segments of the E. coli genome can be deleted, modified or replaced. These powerful tools for genome manipulation create not only marker-less but also scar-less deletions and can therefore be made repeatedly without creating foci for further undesirable genetic events.

The order of events is then expected to be: bacteria find host cell surface, Inv adheres and induces internalization. Bacteria are then contained in vacuoles. OriV replication or other origin of replication turns on by a stress promoter and immunogen DNA is transcribed from an increasing number of copies as TrfA reinitiates multiple replication forks. HlyA destroys the vacuolar membrane and bacteria escape but are slowly killed by limiting nutrients and by oriV-replication, creating multiple replication forks that interfere with normal oriC chromosomal replication. Disintegrating bacteria would then release DNA and/or RNA to be transcribed, spliced and translated by the eukaryotic host. Resulting proteins or peptides then enter the antigen presentation pathway.

To re-engineer the genome in presence of a restriction system, a r⁻m⁺ MDS will be grown in parallel with the bactofection strain. Recognition sites in regulatory regions (AT-rich) will be avoided to minimize effects on gene expression, which can be monitored by genechip expression experiments.

Among the embodiments of the present invention is a Shigella flexneri having a reduced genome. Recently, the complete genome sequence of Shigella flexneri 2a strain 2457T was determined. (The sequenced strain was redeposited at the American Type Culture Collection, as accession number ATCC 700930.) The genome of S. flexneri consists of a single-circular chromosome of 4,599,354 base pairs (bp) with a G+C content of 50.9%. Base pair 1 of the chromosome was assigned to correspond with base pair one of E. coli K-12 since the bacteria show extensive homology. The genome was shown to contain about 4082 predicted genes with an average size of 873 base pairs. The S. flexneri genome exhibits the backbone and island mosaic structure of E. coli pathogens albeit with much less horizontally transferred DNA and lacks 357 genes present in E. coli. (See, Perna et al., (2001) Nature, 409:529-533. The organism is distinctive in its large complement of insertion sequences, several genomic rearrangements, 12 cryptic prophages, 372 pseudogenes, and 195 Shigella specific genes. The completed annotated sequence of S. flexneri was deposited at GenBank accession number AE014073 which is incorporated herein by reference. (See also “Complete Genome Sequence and Comparative Genomics of Shigella flexneri Serotype 2A strain 2457T”, Wei et al., (2003) Infect. Immun. 71:2775-2786.) It is striking to note that based on its DNA sequence, Shigella is phylogenetically indistinguishable from E. coli.

As is readily apparent from this disclosure, having the S. flexneri sequence in hand, its genome may be readily reduced using the methods and gene selection paradigms discussed herein. A reduced genome Shigella may be useful as a bactofection vector, for the expression of heterologous (recombinant) proteins or other useful nutrients for reasons discussed herein with respect to reduced genome E. coli (e.g. live vaccine). Another use for reduced genome Shigella or for that matter any invasive bacteria susceptible to the deletion methods of the present invention, such as Salmonella, is as a vehicle for the display or presentation of antigens for the purpose of inducing an immune response from a host. Such an engineered Shigella could, for example, have genes responsible for virulence deleted from the organism while maintaining other genes such as those encoding antigenic determinants sufficient to induce an immune response in a host and preferably a mucosal immune response in the intestinal wall of a host. Using this sequence information, its genome may be readily reduced using the method and gene selection paradigm described herein.

Shigella flexneri is potentially well suited for this strategy in that its virulence determinants have been characterized and have been localized to a 210-kb “large virulence (or Invasion) plasmid” whose nucleotide sequence has been determined and has been deposited as GenBank Accession No. AF348706 which is incorporated herein by reference. (See also Venkatesan et al. Infection and Immunity (May 2001) 3271-3285).

The deleted Shigella invasion plasmid may be introduced into a reduced genome E. coli thereby allowing efficient expression of certain Shigella invasion plasmid genes capable facilitating entry of the reduced genome E. coli into the target animal cell. The invasion plasmid may also be engineered to delete harmful genes from the plasmid such as the genes encoding the ShET2 enterotoxin, and those responsible for vacuole disruption. Preferred candidate genes for removal from the invasion plasmid include all IS elements, and genes encoding toxins or other pathogenic proteins not involved in invasion include, for example, the virB gene. The present invention also allows the addition of other genes to the reduced genome E. coli into which the invasion plasmid has been introduced so as to optimize delivery of genes into the desired host cell, including genes of the invasion plasmid outside the invasion locus itself, such as the regulator virF.

### II. Invasion/Bactofection

The term “bactofection” as used throughout this application means delivery of foreign or endogenous DNA or RNA into eukaryotic cells by an invasive bacterium preferably by introducing a eukaryotic expression cassette comprising the desired DNA or RNA and which expresses the DNA or RNA in the eukaryotic cell. Delivery organisms that have been used before the present invention include pathogenic strains Salmonella and Shigella spp, Listeria monocytogenes, Yersinia enterocolitica, Vibrio cholerae, Mycobacterium bovis and Bacillus anthracis and their genomes may be reduced according to the present invention

Invasion capability can be supplied by any mechanism employed by invasive bacteria, like that of Yersinia and Listeria (single “invasin” or “internalin” protein), or Shigella and Salmonella (multiple effectors dependent on type III secretion to deliver the signal triggering uptake of the bacteria into the target cell). Invasion mechanisms have recently been reviewed in Cossart, P., and P. J. Sansonetti 2004. Science 304:242-248. In general, bacterial invasion proteins gain access to the interior of the target cell and subvert host-signaling systems to reorganize the cytoskeleton and bring about engulfment of the bacterium. Other mechanisms exist, used by microbes and parasites (Sibley, L. D. 2004 Science 304:248-253).

Shigella and Listeria replicate in the cytosol, and need IpaB or Listeriolysin (escape proteins) to enable them to break out of the vacuoles. Once in the cytosol, these species are able to spread laterally into neighboring cells by actin-based motility; spreading could amplify the immunogenic signal further, although inability to spread might usefully limit the persistence of the delivery bacteria. Preferably, bactofection agents should not persist in humans for more than a few days and should not be shed into the environment.

There are several advantages in using bacterial delivery systems for vaccination. While soluble antigens are poorly antigenic, a direct delivery by bacteria allows any engineered molecule to be presented efficiently. The bacterial delivery system also insures correct protein folding required for proper exposure of the epitope, in the case where it is the protein product rather than RNA that is delivered.

Where vaccination is the desired result, bacterial delivery preferentially targets the mucosal immune system by oral or intranasal or transdermal delivery, (all three routes elicit an immune response at all mucosal membranes). As used herein, “invasive bacteria” are bacteria that are capable of delivering eukaryotic expression cassettes to animal cells or animal tissue. “Invasive bacteria” include bacteria that are naturally capable of entering the cytoplasm or nucleus of animal cells, as well as bacteria that are genetically engineered to enter the cytoplasm or nucleus of animal cells or cells in animal tissue.

Different bacteria replicate in different places inside the host cell. For example, Yersinia and Salmonella replicate in the vacuole created at invasion. Where vaccination is the desired result, delivery of proteins to the vaculolar membrane could direct them into the antigenic pathway (expressed on the surface of antigen-presenting cells along with MHC). SipB/IpaB are able to fuse membranes and could form the pore for delivery of the immunogen into the correct membrane. This process might involve the Golgi or the endoplasmic reticulum of the target cell.

A. Expression Cassettes

The individual elements within the expression cassette can be derived from multiple sources and may be selected to confer specificity in sites of action or longevity of the cassettes in the recipient cell. Such manipulation can be done by any standard molecular biology approach.

A typical expression cassette is composed of a promoter region, a transcriptional initiation site, a ribosome binding site (RBS), an open reading frame (orf) encoding a polypeptide, optimally with sites for RNA splicing (in eukaryotes), a translational stop codon, a transcriptional terminator and post-transcriptional poly-adenosine processing sites (in eukaryotes). The promoter region, the RBS, the splicing sites, the transcriptional terminator and post-transcriptional poly-adenosine processing sites are different in eukaryotic expression cassettes than those found in prokaryotic expression cassettes. These differences prevent expression of prokaryotic expression cassettes in eukaryotic cells and vice versa.

These cassettes usually are in the form of plasmids, and contain various promoters well known to be used for driving expression of genes in animal cells, such as the viral derived SV40, CMV and RSV promoters. Tissue-specific promoters, such as the beta-casein promoter (selectively active in mammary tissue); the phosphoenolpyruvate carboxykinase promoter (active in liver, kidney, adipose, jejunum and mammary tissues); the tyrosinase promoter (active in lung and spleen cells, but not testes, brain, heart, liver or kidney); the involucrin promoter (active in differentiating keratinocytes of the squamous epithelia) and the uteroglobin promoter (active in lung and endometrium) can be used.

Additional genetic elements on the plasmid may include but are not limited to enhancers, a polyadenylation signal, the inverted repeats from adeno-associated virus, a restriction enzyme recognition site.

Amplifiable copy number plasmids, such as pBAC3, see below, may carry the immunogen gene or genes, which remain single-copy until replication is induced. In the final version of the bactofection strain, the immunogen gene(s) and replication-amplifying segment of the plasmid may be designed to be incorporated into the bacterial genome if it is desired to eliminate the need for any plasmid or selectable marker. Induction of replication copies of a chromosomal segment will prevent normal oriC replication by producing multiple replication forks and thus limit viability in the host.

Amplification and expression can be controlled by promoters that are induced on entering the mammalian target cells. DNA genechip experiments monitor gene expression of internalized bacteria, enabling the identification of useful promoters that are induced in the intracellular environment (Runyen-Janecky, L. J., and S. M. Payne. 2002. Infect. Immun. 70:4379-88.). Invasion-inducible promoter(s) will be added to trfA (to drive DNA amplification) and the reporter or immunogen gene (to drive transcription). A characterized promoter in Shigella like that of sitB, encoding an iron-uptake protein induced by iron-limiting conditions inside human cells, or that of uhpT, induced by glucose-6-phosphate inside human cells, could be used. These promoters have the advantage of being characterized, but a stress-induced promoter would be preferable and may be found by the genechip scan. The interior of a human cell is a stressful environment for bacteria in many respects. A further alternative is to synthesize a promoter of novel design with a transcription factor-binding site for a stress-induced sigma factor e.g. RpoS or RpoE.

In one preferred embodiment, the elements for invasion and subunit vaccine delivery are assembled in a BAC referred to a pBAC3. Once it is shown that all the desired elements are working, for example oriV, inv, and the vaccine candidate gene, all with the appropriate regulatory sequences can be transferred into the lambda attachment site attB in the MDS chromosome. This site is chosen as one known to accept phage-sized inserts (up to 50 kb) without negative effects on the host. Inv would be expressed at the time of infection or constitutively if that is not lethal. Expression of the oriV replication protein TrfA (integrated at a separate locus) and the vaccine gene would be turned on upon invasion of host cell. Clean insertion with no other changes can be confirmed by DNA chip hybridization.

B. Restriction-Modification Systems

In one preferred embodiment, an exogenous restriction/modification system to defend against horizontal DNA transfer can be added to the clean genome strains of the present invention. In a preferred embodiment, this may be achieved by adding such restriction/modification system such as PvuII restriction endonuclease and methylase not normally found in the strains of the present invention so that the MDS genome is protected (methylated in the appropriate pattern) but any incoming DNA will be destroyed by the restriction enzyme cutting at recognition sites that are not methylated. The methylase gene must be inserted first and preferably constitutively expressed to protect the genome when the restriction enzyme gene is introduced. From the large number of restriction enzymes and methylases that have been cloned in E. coli for commercial purposes, one or more systems from non-pathogenic organisms may be chosen that is not normally found in mammalian gut, so that the chance of incoming DNA being already protected is remote. To re-engineer the genome in presence of a restriction system, it is necessary to make a r⁻m⁺ MDS in which to propagate constructs. This can easily be done in parallel within the bactofection strain. Recognition sites in regulatory regions (AT-rich) will be avoided to minimize effects on gene expression, which can be monitored by genechip expression experiments.

Among the advantages of the bacterial strains of the present invention are that it lacks all known or potential cryptic virulence genes that might contribute to pathogenicity, so that the risk of recombination or a combination of several recombinations producing any new pathogenic function on addition of invasion/immunogen gene(s) is very low. In addition, the engineered deletions are stable and cannot revert except by recombination with exogenous DNA; deletion of all IS elements and other recombinogenic elements minimize the possibility of recombination and/or horizontal transfer of virulence genes with commensals or other pathogens; deletion of IS and phage elements will prevent undefined genetic alterations during passage, a troublesome problem with current attenuated vaccine strains; no drug resistance markers or plasmids will remain in the delivery strain, for example, provision of a minimal invasion locus from Shigella invasion locus Salmonella invasion genes or the invA gene of Yersinia pseudotuberculosis or genes encoding any other bacterial invasion system or partial system, genes stabilize the host cell entry phenotype in MDS42 and MDS43 without further pathogenicity; MDS42 and MDS43 are derivatives of E. coli K-12, a well-tolerated, generally recognized as safe, commensal; and MDS42 and other E. coli derivatives, such as MDS43, are entirely appropriate for oral delivery. Reduced genome strain MDS42 was produced using methods as described in International Patent Publication No. WO 2003/070880 by deleting the endA gene from parental strain MDS41.

The resulting bacterial strains are used to deliver multivalent nucleic acid based vaccines making it possible to produce an orally administered vaccine that is effective against multiple pathogens. The bacterial strains may also be used for gene therapy or biochemical therapy, such supplying a missing or mutant metabolic function or a molecule that controls a function, such as a transcription factor. Moreover, the bacterial strains may be used for any delivery purpose where genome stability is important, or assurance that no genomic elements will be transferred is important.

### III. Heterologous Genes/Antigens

In the present invention, the live invasive bacteria with clean genome can deliver either a heterologous or endogenous gene into animal cells. As used herein, “heterologous gene” means a gene encoding a protein or fragment thereof or anti-sense RNA or catalytic RNA, which is foreign to the recipient animal cell or tissue, such as a vaccine antigen, immunoregulatory agent, therapeutic agent or transcription factor. An “endogenous gene” means a gene encoding a protein or part thereof or anti-sense RNA or catalytic RNA which is naturally present in the recipient animal cell or tissue.

Where vaccination is the desired result, single or multiple expression cassettes can be delivered using live invasive bacteria with clean genome that express any combination of viral, bacterial, parasitic antigens, or synthetic genes encoding all or parts or any combination of viral, bacterial, parasitic antigens.

Where transfection of eukaryotic cells in vitro is desired, single or multiple expression cassettes can be delivered using live invasive bacteria with clean genome that express any combination of foreign or endogenous genes such as transcription factors of animal origin.

A. Vaccination

Currently available attenuated bacterial strains that are generally regarded as safe for vaccine use have been derived from natural pathogens isolated by repeated application of empirical methods of attenuation involving many steps of random mutagenesis followed by tests. Unfortunately these strains are very poorly characterized by current genomically based scientific standards. But if, as expected, they resemble the sequenced genomes of E. coli, Salmonella and Shigella, they will contain hundreds of genes for toxins, fimbrae, invasins, Type III secretion systems, phage, virulence determinants, and pathogenicity islands plus a large array of mobile genetic elements capable of promoting genome instability by moving DNA segments around.

Mounting evidence also suggests that the phenomenon of horizontal transfer of genetic elements has been underappreciated in the context of vaccine development, although acquisition of multiple antibiotic resistance by the horizontal transfer mechanism has resulted in a resurgence of infectios diseases (e.g., typhoid fever and tuberculosis that are now refractory to drugs).

Among the advantages of the present invention are that it applicable to essentially any bacterial vaccine vector regardless of its intended use. For example, there remains an acute need for a single-dose typhoid vaccine that is also safe and effective. Utilizing teachings of the instant specification, clean genome strains of Salmonella (or E. coli) may be engineered to elicit protective immunity to typhus. In addition, these stains could be engineered further to elicit immunity to any of a variety of other viral or microbial pathogens including select agents by inserting relevant genes encoding immunogens that elicit protective immunity. These could be included by direct integration into the bacterial chromosome or as an expressible DNA in a vector such as a plasmid or bacterial artificial chromosome (BAC) that is delivered into a cell in a clean genome strain specifically designed to deliver such a vaccine. In this way, it is possible to elicit protective immunity against typhoid in addition to other pathogens such as hepatitis B by using a single vaccine. The clean genome approach affords greater margin of predictable safety for both the vaccine and the environment when compared to other types of vaccines. Bacterial strains developed according to teachings of the instant invention have inter alia the following features: 1) ability to deliver multiple vaccine antigens; 2) defined and stable attenuating mutations; 3) inability to transfer or receive genetic information from the environment; and 4) only those traits necessary for vaccine efficacy are present. In addition, these bacterial strains preferably can deliver vaccines orally.

Plasmid BAC constructs or the like containing eukaryotic expression systems can be delivered into mammalian cells using the bacteria of the present invention, using plasmids bearing genes encoding therapeutic or antigenic molecules under controlled regulation. Whereas soluble antigens are poorly antigenic, direct delivery by bacteria allows any engineered molecule to be presented efficiently, and allows engineering of the plasmid construct to ensure correct protein folding to expose the relevant epitope or epitopes. Delivery organisms that have been used include pathogenic strains Salmonella and Shigella spp, Listeria monocytogenes, Yersinia enterocolitica, Y. pseudotuberculosis, Vibrio cholerae, Mycobacterium bovis and Bacillus anthracis. The advantages of the clean genome strains of the present invention over these strains meet nearly all the desired features and problems described above.

The Multiple Deletion Strains (MDS) of the instant invention can be engineered to fine-tune the desirable properties. Reversion of attenuating mutations can be avoided by using scarless, markerless deletions, especially in combination. Immunogenicity of the MDS itself can be controlled by deletion of all secondary antigen genes that are not essential, and modifying those that are. E. coli bacterial strain K-12 does not make 0- or H-antigen, but does make lipid A which is a good candidate for modification. Deletion of genes encoding fimbriae, flagella, outer membrane receptors for phage attachment, nucleases, secreted proteins (toxins, IgA proteases) can be used to modulate bacterial immunogenicity versus adjuvant effect. The bacteria of the instant invention must survive within the host cell long enough to deliver the antigen, but not persist for more than a few days. Using MDS strain provides exquisite control over the antigenic challenges presented to the mucosal immune system since genes can be added or subtracted at will with the goal of balancing the severity of the challenge against the level of protection required. The delivery bacterial strains of the instant invention are stable and cannot revert and attenuation can be fine-tuned. Once the delivery strain is engineered and ready to be used for vaccine delivery, it carries no drug resistance markers or plasmids. IS elements and recombinogenic elements are removed from the delivery strains and a restriction/modification system may be added. This minimizes the possibility of genetic exchange with commensals or other pathogens. A minimal invasion locus or gene of the delivery strain stabilizes the host cell entry phenotype without pathogenicity. Finally, when E. coli K-12 is used, then its derivatives are entirely appropriate for oral delivery because K-12 is a well-tolerated, generally recognized as safe, commensal.

The natural pathogens from which vaccines have been developed by attenuation are biologically quite complex and require a constellation of virulence elements, probably numbering on the order of 100, to be fully virulent. Empirical methods of attenuation may only inactivate a few of these or simply weaken the bacterial fitness without really eliminating virulence elements per se. The discovery that horizontal transmission of virulence genes may be a significant mechanism in the emergence of new pathogens takes on added significance when a vaccine containing residual virulence genes becomes widely distributed.

Transfer of virulence elements out of a vaccine strain that is widely used, into the normal intestinal flora could convert these normal flora into “pathogens waiting to happen.” That is it could increase their pathogenic potential. Conversely, transfer of genetic information into the vaccine strain from the environment could reverse attenuation by recombination. These considerations dictate that the vaccine strain has the minimum number of potential virulence elements to make it combinatorially difficult to create a pathogen out of it, or from it and the transpositional and recombinational mechanisms that may participate in such combinatorial event should be eliminated to the greatest extent possible.

By way of example, the delivered DNA will drive the expression of SCBaL/M9, a potential HIV vaccine antigen, as described below. Other or multiple immunogens may also be used, including but not limited to those deemed to be useful from other pathogenic organisms or viruses, or tumor virus antigens.

The general approach to the construction of bacterial strains for use in reduced genome or clean genome bactofection delivery according to the present invention is as follows:

A defined reduced genome E. coli strain is engineered to confer immunogen delivery capability on the strain by inserting relevant portions of Shigella virulence plasmid, which confer invasiveness Salmonella invasion genes, the invA gene of Yersinia pseudotuberculosis or genes encoding all or part of any other bacterial invasion system or partial system, to promote bactofection.

Inserting into the strain an expressible immunogen encoding gene (or antigen encoding gene), for example, (SCBaL/M9) into an amplifiable expression system (expression cassette, for example, a BAC) designed to be activated (expressed and preferably replicable) when it is introduced into an eukaryotic cell and which may preferably deliver or expression RNA product in the cell in a form that can be spliced, processed, and translated by the cell.

Eliminating any drug resistance marker in the plasmid intermediates used for assembling the DNA segments in the amplifiable expression system or replacing them with an essential gene selectable marker.

Integrating delivery construct (expression cassette) into the reduced genome chromosome to eliminate the need for a plasmid vector with a selectable marker (although integration of the construct is not necessary for delivery, it is preferred for safety).

The vaccine antigen may be a protein or antigenic fragment thereof from a viral pathogen, bacterial pathogen, or parasitic pathogen or may be a tumor antigen. The vaccine antigen may be encoded by a synthetic gene, constructed using recombinant DNA methods, which encode antigens or parts thereof from viral, bacterial, parasitic pathogens. These pathogens can be infectious in humans, domestic animals or wild animal hosts. The antigen can be any molecule that is expressed by any viral, bacterial, parasitic pathogen prior to or during entry into, colonization of, or replication in their animal host.

The heterologous nucleic acid sequence, or interchangeably, heterologous gene, can encode an antigen, an antigenic fragment of a protein, a therapeutic agent, an immunoregulatory agent, an anti-sense RNA, a catalytic RNA, a protein, a peptide, an antibody, an antigen-binding fragment of an antibody, or any other molecule that can be synthesized in the clean genome strain after appropriate engineering (hormone, lipid, sugar, enzyme, anti-disease drug eg anti-cancer agent) and that is desired for delivery to an animal or animal cell. The heterologous nucleic acid sequences can be obtained from any pathogen virus selected, for example, from the group consisting of influenza virus, respiratory syncytial virus, HPV, HBV, HCV, HIV, HSV, EDBV, FeLV, FIV, HTLV-I, HTLV-II, Ebola virus, Marburg virus, and CMV. These abbreviations are used for these following viruses: HPV, human papilloma virus; HBV, hepatitis B virus; HCB, hepatitis C virus; Lenti viruses, HIV, human immunodeficiency virus; HSV, herpes simplex viruses; FeLV, feline leukemia virus; FIV, feline immunodeficiency virus; HTLV-I, human T-lymphotrophic virus I; HTLV-II, human T-lymphotrophic virus II; CMV, cytomegalovirus. Rhabdoviruses, such as rabies; Picornoviruses, such as poliovirus; Poxviruses, such as Vaccinia; Rotavirus; and Parvoviruses. Examples of protective antigens of viral pathogens include the HIV antigens nef, p24, gp120, gp41, gp160, env, gag, tat, rev, and pol [Ratner et al., Nature 313:277-280 (1985)] and T cell and B cell epitopes of gp120 [Palker et al., J. Immunol. 142:3612-3619 (1989)]; the hepatitis B surface antigen [Wu et al., Proc. Natl. Acad. Sci. USA 86:4726-4730 (1989)]; rotavirus antigens, such as VP4 and VP7 [Mackow et al., Proc. Natl. Acad. Sci. USA 87:518-522 (1990); Green et al., J. Virol. 62:1819-1823 (1988)], influenza virus antigens such as hemagglutinin or nucleoprotein (Robinson et al., supra; Webster et al., supra) and herpes simplex virus thymidine kinase (Whitley et al., In: New Generation Vaccines, pages 825-854). In the case of HW, the antigens can be from any structural, accessory or regulatory gene, and includes combinations or chimeras of such genes in multiple or single replicons. In a preferred embodiment, the heterologous gene encodes at least one antigen or antigenic fragment from each of the HIV genes env, gag, pol, nef, tat, and rev.

The bacterial pathogens, from which bacterial antigens may derive include any pathogenic bacterium, including but not limited to, Mycobacterium spp., Helicobacter pylori, Salmonella spp., Shigella spp., E. coli, Rickettsia spp., Listeria spp., Legionella pneumoniae, Pseudomonas spp., Vibrio spp., Borellia burgdorferi, Bacillus anthacus, Bordetlla, Streptococcus, Staphylococcus, Yersinia, Corynebacteria, Clostridium, Enterococcus, Neisseria, Campylobacter, Bacteroides, Serratia, Treponema, and Cyanobacter.

Examples of protective antigens (antigens that give rise to protective immunity) of bacterial pathogens include the Shigella sonnei form 1 antigen [Formal et al., Infect. Immun. 34:746-750 (1981)]; the 0-antigen of V. cholerae Inaba strain 569B [Forrest et al., J. Infect. Dis. 159:145-146 (1989); protective antigens of enterotoxigenic E. coli, such as the CFA/I fimbrial antigen [Yamamoto et al., Infect. Immun. 50:925-928 (1985)] and the nontoxic B-subunit of the heat-labile toxin [Clements et al., Infect. Immun. 46:564-569 (1984)]; pertactin of Bordetella pertussis [Roberts et al., Vacc. 10:43-48 (1992)], adenylate cyclase-hemolysin of B. pertussis [Guiso et al., Micro. Path. 11:423-431 (1991)], and fragment C of tetanus toxin of Clostridium tetani [Fairweather et al., Infect. Immun. 58:1323-1326 (1990)].

B. Shiga Toxins

Shiga toxins encoded are highly potent protein toxins belonging to a family of ribosome-inhibiting proteins. In human target cells, protein synthesis is shut off. They are secreted by S. dysenteriae and certain STEC strains (Shiga toxin producing E. coli). On infection by these pathogens, the secreted toxins can complicate diarrhea into a life threatening disease progressing to kidney failure and damage to the central nervous system. No treatments are currently available to halt this progression. The usual treatments for diarrheal disease, antibiotics and antidiarrheal agents, do not prevent toxin activity, and may even exacerbate it. To date, there is no effective vaccine and candidates are difficult to test due to the lack of a truly relevant animal model.

Current approaches to prophylaxis and treatment of STEC infection and (hemolytic uremia syndrome) HUS include vaccines to prevent attachment and colonization by STECs, and passive therapies aimed at binding/inactivating Stxs. Intimin, the bacterial adhesin, and the toxin B subunit that binds receptors on mammalian cells have been used as immunogens in mice. Recently, Capozzo et al. reported that an injected DNA vaccine based on an active site-deleted Stx2 gene raised protective immunity in mice. Stx1 with amino acid substitutions at key active site residues have also produced protective immunity to toxin challenge in mice, again administered by injection.

Among the passive therapies are Stx toxoid, monoclonal antibodies to Stxs (including humanized versions), neither of which has yet been approved for human use. Non-antibody agents that mimic the glycolipid receptor ligand for Stxs has been devised to tightly bind free toxin in the gut lumen. Synsorb (a trisaccharide glycoside attached to diatomaceous silica) has been used to treat HUS. In a phase II human trial, though safe, it did little to divert the course of toxicity. Other receptor mimic multivalent carbohydrate ligands, have been tested in mice by subcutaneous injection. Protective activity was obtained, but the compounds are expensive as well as requiring injection. Multivalent synthetic polymers (receptor mimics) reportedly reduced both intestinal and circulating StxA when fed to mice. A recombinant LPS has even been expressed on the surface of E. coli and was shown to bind Stx and protect mice effectively from a lethal toxin dose, but the strain used has all the potential instability problems.

The Shiga toxin genes are encoded on prophage in the STEC genomes. Since phage induction to the lytic cycle can be stimulated by quinolone antibiotics, these drugs cannot be used to clear STEC infections without the risk of increasing toxin production. Toxin expression is regulated by phage late transcription and antitermination by the phage Q protein. In any case, by the time the infectious agent is identified, toxins are already circulating. In addition, antibiotic resistance is now being found with increasing frequency in STECs.

A preferred embodiment of the invention is illustrated by a single-dose typhoid vaccine that is also safe and effective. A clean genome strain such as E. coli MDS41 or any other MDS strain which meets the criteria described herein for suitably as a vaccine may be engineered such that it elicits protective immunity to typhoid. Genes encoding the relevant antigens can be included by direct integration (in an expression cassette) into the bacterial chromosome or as a DNA vaccine that is delivered by a clean genome strain specifically designed to deliver such a vaccine. In this way, it should be possible to elicit protective immunity against typhoid in addition to other pathogens such as hepatitis B virus by using a single vaccine. Thus, the clean genome approach disclosed under the instant invention affords a much greater margin of safety for both the vaccine and the environment.

One of the major advantages of a clean genome organism according to the present invention is to provide a clean, minimal genetic background into which DNAs may be introduced to not only allow expression of a desired molecule, but it also affords the opportunity to introduce additional DNAs into the clean background to provide a source of molecules capable of optimizing expression of the desired agent or optimizing the host response to the agent.

In one preferred embodiment of the instant invention, constructs are developed to express mStx2 either as soluble subunit vaccines (i.e. vaccines based on delivery of single proteins) from MDS43, the prototype clean-genome strain, or from a plasmid suitable for eukaryotic cell expression (DNA vaccine).

Shiga toxins belong to a family of AB subunit protein toxins including ricin and cholera toxin. Much of Stx biology is known, enabling a rational mutation strategy to be designed. Stxs consist of an A subunit bearing the catalytic site, and five B subunits which form the receptor-binding moiety. The crystallographic structures of Stx, Stx1 and Stx2 are known. A and B are non-covalently attached. The A subunit consists of A1 and A2 separated by a protease-sensitive site, and with a disulphide bond linking the two portions. A2 attaches the A protein to the B-pentamer. The active site resides in the A1 portion. The immunogen for the clean-genome vaccine will be based on this A1 polypeptide.

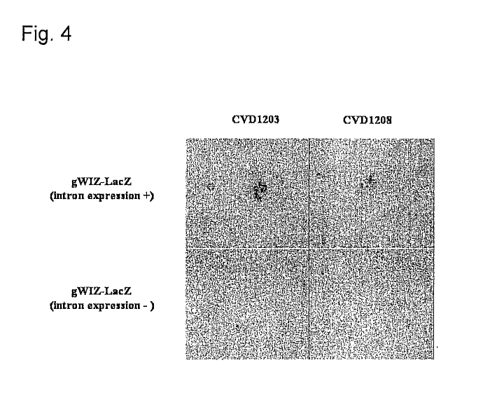

Strictly, the term “Stx” refers specifically to the Shiga toxin of Shigella dysenteriae, whereas Stx1 and Stx2 are toxins of the E. coli pathogens. Either or both may be found in individual isolates. Stx1 and Stx are almost identical, but only about 56% identical with Stx2, though the active site is highly conserved in all Stxs (see FIG. 4). Several variants of Stx2 have been identified whose toxic characteristics vary. For example, Stx2 from enterohemorrhagic E. coli (EHEC) 0157:H7, a highly virulent strain which has been most frequently the cause of HUS. In the text below, as in common usage, the term Stx has also been used to refer generically to the entire Shiga toxin family and mStx to indicate mutant Stx2.

Production of Stx2 is controlled by induction of the prophage on which the A and B genes are encoded together in an operon, and transcription is induced when the prophage enter the lytic cycle. Expression of the lytic protein genes downstream is coupled to Stx transcription, and phage-mediated bacterial cell lysis is an obvious way for the toxin to be released [35, 56]. The lysis genes R, S and R7 from lambda expressed from an inducible promoter are used in the embodiments of the instant invention to bring about bacterial lysis after invasion.

While it is likely that the prophage is induced by changing environmental signals upon host cell invasion, the phage regulation circuits are complex and the signals as yet undefined. Rather than using phage regulation, the promoter of the uhpT gene identified as inducible may be used in the embodiments of the instant invention.

The uhpT gene encodes a hexose phosphate transporter and is induced in vitro by glucose-1-phosphate, which is present in the host cell cytosol but not in bacteria. MDS43 contains an ortholog of this gene. Thus, it is possible to insert the lambda SRRZ genes into the genome replacing uhpT, or to add the promoter and genes to pBAC3-invA. Expression of the lysis genes may be tested by addition of glucose-1-phosphate to a growing bacterial culture, when visible cell lysis should rapidly follow. Insertion of this “suicide” lysis cassette into MDS43 would also serve to limit the time the bacteria remain viable in the host after invasion, meeting a concern of the regulatory agencies about bacterial persistence.

Stx2 A-subunit protein is synthesized with a signal sequence that could target it to the E. coli periplasm. The A and B subunits are assembled with a disulfide bond forming the AB5 holotoxin. The B-pentamer forms the receptor attachment structure. The holotoxin is secreted or released by phage lysis into the lumen of the intestine or into a vacuole of an invaded host cell. The toxin can cross the intestinal barrier via M cells, gaining access to the blood and lymphatic system. Circulation enables the toxin to reach cells bearing the glycolipid Gb3 (globotriaosylceramide) receptors to which it specifically attaches. Endothelial cells lining the microvasculature of the kidney and CNS are targeted because of the high levels of Gb3 receptors on their surfaces.

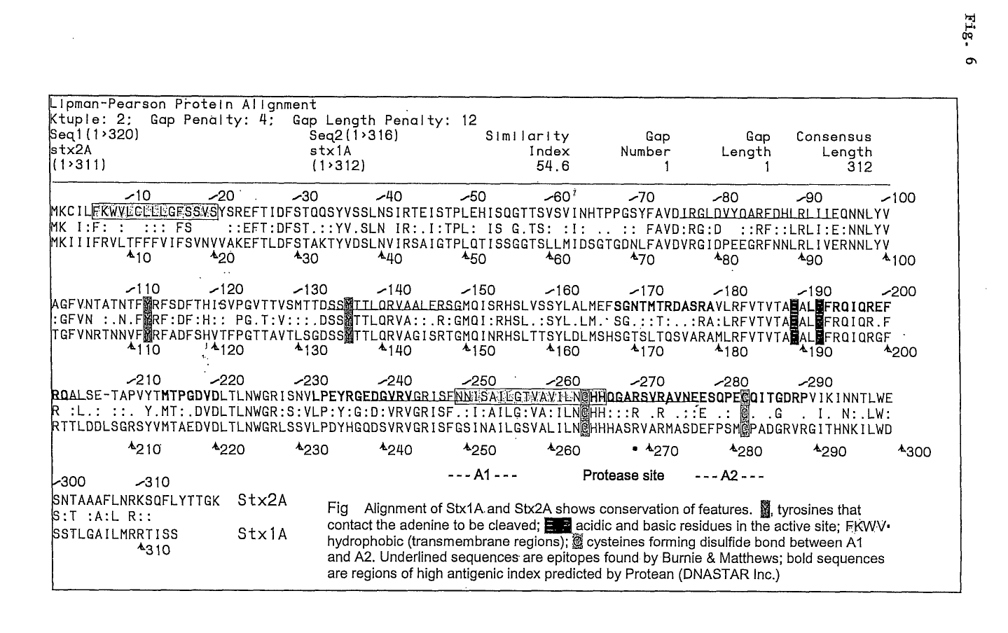

Receptor-bound toxin is internalized mainly by clathrin-mediated endocytosis. It enters the Golgi and is transported through to the ER in a process known as retrograde transport [48]. During transport the A and B proteins are separated by cleavage of A by the eukaryotic protease furin and by disruption of the disulphide bond (FIG. 6). A1 is then transported into the cytosol, probably using the internal transmembrane domain (FIG. 6). In the cytosol its highly potent N-glycosidase activity cleaves a specific adenine residue from mammalian 28S ribosomal RNA, lethally blocking protein synthesis.

A mutant Stx2 toxin from which the active site of the A subunit was deleted (mStx2 AA) has been described that, when administered as DNA vaccine in mice, elicits a potent humoral response that protects against lethal Stx2 challenge. Based on these protection studies, this mStx was selected to facilitate our own proof of concept mouse studies with MDS43. Two strains will be constructed for this effort. The first expresses the mStx2 AA in MDS43 pBAC3-invA strain as a prokaryotically expressed subunit protein.

To increase production of soluble mSTX2 protein and thus, improve invasiveness of bacterial strains of the instant invention into the mammalian host cells, the copy number of the prokaryotic or eukaryotic expression cassettes may be increased by using, for example, genetic elements that insure high copy number during expression cassettes replication. For example, a second inducible high-copy replication origin can be added to an expression cassette. The origin can then be activated by an inducible replication protein such as, for example, TrfA203.

The parasitic pathogens, from which the parasitic antigens are derived, include but are not limited to, Plasmodium spp., Trypanosome spp., Giardia spp., Babesia spp., Entamoeba spp., Eimeria spp., Leishmania spp., Schistosome spp., Brugia spp., Fasciola spp., Dirofilaria spp., Wuchereria spp., and Onchocera spp.

Examples of protective antigens of parasitic pathogens include the circumsporozoite antigens of Plasmodium spp. [Sadoff et al., Science 240:336-337 (1988)], such as the circumsporozoite antigen of P. bergerii or the circumsporozoite antigen of P. falciparum; the merozoite surface antigen of Plasmodium spp. [Spetzler et al., Int. J. Pept. Prot. Res. 43:351-358 (1994)]; the galactose specific lectin of Entamoeba histolytica [Mann et al., Proc. Natl. Acad. Sci. USA 88:3248-3252 (1991)], gp63 of Leishmania spp. [Russell et al., J. Immunol. 140:1274-1278 (1988)], paramyosin of Brugia malayi [Li et al., Mol. Biochem. Parasitol. 49:315-323 (1991)], the triose-phosphate isomerase of Schistosoma mansoni [Shoemaker et al., Proc. Natl. Acad. Sci. USA 89:1842-1846 (1992)]; the secreted globin-like protein of Trichostrongylus colubriformis [Frenkel et al., Mol. Biochem. Parasitol. 50:27-36 (1992)]; the glutathione-S-transferases of Fasciola hepatica [Hillyer et al., Exp. Parasitol. 75:176-186 (1992)], Schistosoma bovis and Shistosoma japonicum [Bashir et al., Trop. Geog. Med. 46:255-258 (1994)]; and KLH of Schistosoma bovis and Shistosoma japonicum [Bashir et al., supra].

C. In Vitro Gene Delivery

The clean genome bacteria of the invention are also useful in methods of gene delivery to animal cells in vitro. The animal cells can be further cultured in vitro, and the cells carrying the desired genetic trait can be enriched by selection for or against any selectable marker introduced to the recipient cell at the time of bactofection. Such markers may include antibiotic resistance genes, selectable cell surface markers, or any other phenotypic or genotypic element introduced or altered by bactofection. Use of the clean genome bacteria of the invention in methods of bactofection provides several advantages. Surprisingly, a significant increase in bactofection efficiency is observed using the clean genome bacteria of the invention. As used herein, the term “bactofection efficiency” refers to the percentage of target cells within a population of target cells, that contain a nucleic acid molecule introduced by bactofection. Moreover, the use of clean genome bacteria allows the introduction of multiple genes into eukaryotic cell cultures via a very gentle method.

In one embodiment, the invention comprises a method for introducing and expressing nucleic acid or gene in an animal cell (e.g. a mammalian cell) comprising: (a) transforming at least one invasive clean genome bacterium with a vector comprising a eukaryotic expression cassette, said expression cassette comprising said gene to form at least one transformed bacterium; and (b) infecting the animal cell with said transformed bacterium. In a related embodiment, the nucleic acid or gene is expressed at detectable levels in the animal cell. In another embodiment, the animal cells are cultured in vitro.

An “invasive bacterium” herein is a bacterium naturally capable of entering the cytoplasm or nucleus of animal cells, as well as bacterium that are genetically engineered to enter the cytoplasm or nucleus of animal cells.

In a related embodiment, the vector comprises a first and second origin of replication. The first origin of replication is a low-copy number origin of replication such as oriS. In yet another embodiment, the second origin of replication is an inducible high-copy number origin of replication such as oriV. In one embodiment, the high-copy number origin of replication is under the control of an arabinose promoter. In another embodiment, the high-copy number origin of replication is regulated by a TrfA encoded by a gene under the control of an arabinose promoter.

Surprisingly, it has been determined (see Example 11) that freezing transformed reduced genome bacteria in aqueous glycerol solution prior to infection significantly increases bactofection efficiency. Accordingly, in a preferred embodiment, the invention comprises a method for introducing and expressing nucleic acid or gene in an animal cell (e.g. a mammalian cell) comprising: (a) transforming at least one invasive clean genome bacterium with a vector comprising a eukaryotic expression cassette, said expression cassette comprising said gene to form at least one transformed bacterium; (b) freezing said transformed bacterium in an aqueous glycerol solution; and (c) infecting the animal cell with said transformed bacterium. The aqueous glycerol solution may be about 1%, about 5%, about 10%, about 11%, about 12%, about 13%, about 14%, about 15%, about 16%, about 17%, about 18%, about 19%, about 20%, or about 25% weight/weight (w/w) glycerol, although aqueous glycerol solution having about 15% w/w glycerol is preferred. The transformed bacterium may be frozen to a temperature of about 0° C., about −5° C., about −10° C., about −15° C., about −20° C., about −25° C., about −30° C., about −35° C., about −40° C., about −45° C., about −50° C., about −55° C., about −60° C., about −65° C., about −70° C., about −75° C., about −80° C., about −85° C., about −90° C., about −95° C., or about −100° C., although freezing to a temperature of about −80° C. is preferred. Other cell-permeating cryoprotective agents such as dimethyl sulfoxide, are also contemplated for use in the method.

In a related embodiment, a method for preparing a reduced genome bacterium for bactofection is provided comprising (a) providing a vector comprising a first origin of replication, a second origin of replication, and a eukaryotic expression cassette, said expression cassette comprising a nucleic acid or gene (b) transforming at least one invasive reduced genome bacterium with the vector to form at least one transformed bacterium and (c) freezing said transformed bacterium in aqueous glycerol solution. Also provided is a reduced genome bacterium prepared by this method. In a preferred embodiment, the reduced genome bacterium prepared by this method comprises a vector comprising a eukaryotic expression cassette comprising a nucleic acid or gene, wherein said nucleic acid or gene is under the control of a cardiac-specific promoter. In a related embodiment, the nucleic acid or gene is selected from vascular endothelial growth factor (VEGF) 1; VEGF 2; fibroblast growth factor (FGF) 4; endothelial nitric oxide synthase (eNOS); heme oxygenase-1 (HO-1); extracellular superoxide dismutase (Ec-SOD); heat shock protein 70 (HSP70); Bc1-2; hypoxia-inducible factor 1 (HIF-1) alpha; sarcoplasmic reticulum Ca²⁺-adenosinetriphosphatase (SERCA); sarcoplasmic reticulum Ca²⁺-adenosinetriphosphatase-2 (SERCA2); and sulfonylurea receptor-2 (SUR2).

Any mammalian cell may be used in the methods, including, without limitation, human, bovine, ovine, porcine, feline, buffalo, canine, goat, equine, donkey deer, primate and murine. The most preferred mammalian cell is a human cell. Particularly preferred mammalian cells are fibroblasts, non-limiting examples of which include IMR90 fetal fibroblasts, postnatal foreskin fibroblasts, and adult dermal fibroblasts. Also preferred are mammalian stem cells, including embryonic stem cells, which have the capacity to give rise to every cell type (i.e. they are totipotent) and adult stem cells such as hematopoietic stem cells, mesenchymal stem cells, stromal stem cells, neural stem cells, myoblasts, and cardiac stem cells. Mammalian stem cells may be isolated from embryonic tissue, bone marrow, umbilical cord blood, somatic tissue, or may be generated from somatic mammalian cells. Also preferred are HeLa cells, human embryonic kidney (HEK) 293 cells and mouse and human cardiomyoctes.

In one preferred embodiment, the mammalian cell used in the methods is a cardiomyocyte. Cardiac cells, particularly cardiomyocytes, are relatively difficult to transfect or infect by traditional methods. The present invention provides a method for efficient gene or nucleic acid delivery to cardiomyocytes. In such an embodiment, it may be desirable to place the gene or nucleic acid in the eukaryotic expression cassette under the control of a cardiac specific promoter. Suitable cardiac-specific promoters include, without limitation, an α-myosin heavy chain promoter, a β-myosin heavy chain promoter, a myosin light chain-2v promoter, a myosin light chain-2a promoter, cardiomyocyte-restricted cardiac ankyrin repeat (CARP) promoter, cardiac α-actin promoter, ANP promoter, BNP promoter, cardiac troponin C promoter, cardiac troponin T promoter, and skeletal α-actin promoter. In a related embodiment, the gene or nucleic acid to be delivered to a cardiomyocyte is selected from the group consisting of: vascular endothelial growth factor (VEGF) 1; VEGF 2; fibroblast growth factor (FGF) 4; endothelial nitric oxide synthase (eNOS); heme oxygenase-1 (HO-1); extracellular superoxide dismutase (Ec-SOD); heat shock protein 70 (HSP70); Bcl-2; hypoxia-inducible factor 1 (HIF-1) alpha; sarcoplasmic reticulum Ca²⁺ ATPase (SERCA); sarcoplasmic reticulum Ca²⁺-adenosinetriphosphatase-2 (SERCA2); and sulfonylurea receptor-2 (SUR2).

In a preferred embodiment, the gene to be introduced and expressed in the mammalian cell is a factor (e.g. transcription factor) which, in combination with one or more additional factors, is sufficient to generate pluripotent stem (iPS) cells from somatic mammalian cells. The induction of iPS cells from somatic cells is described in Takahashi et al. Cell 131:861-872 (2007), Nakagawa et al., Nat. Biotechnol. 26:101-106 (2008) and Yu et al. Science 318:1917-1920 (2007). Takahashi et al. reports the induction of iPS cells from mouse fibroblasts and adult human fibroblasts following retrovirus-mediated transduction of human Oct3/4, Sox2, Klf4 and c-Myc. Nakagawa et al. reports the induction of iPS cells from mouse and human fibroblasts following retrovirus-mediated transduction of human Oct3/4, Sox2 and Klf4. Nakagawa reports that certain members of the Sox and Klf transcription factor families can substitute for Sox2 and Klf4. Specifically, Sox1, Sox3 and Sox15 were able to substitute for Sox2 and Klf1, Klf2 and Klf5 were able to substitute for Klf4. Yu et al. reports the induction of iPS cells from human IMR90 fetal fibroblasts and from human newborn (postnatal) foreskin fibroblasts. Noteably, the iPS cells generated in each study had human (or mouse) embryonic stem (ES) cell morphology, had a normal karyotype, expressed cell surface markers and genes characteristic of human (or mouse) ES cell, and were capable of multilineage differentiation.

As used herein, “induced pluripotent stem (iPS) cell” refers broadly to a cell which is pluripotent, i.e. a cell which has the capacity to give rise to two or more tissues or a type of tissue which is distinct from the originating cell, and which has been generated from a somatic cell. A somatic cell is defined herein as a diploid cell of any tissue/structural type that does not contribute to the propagation of the genome beyond the current generation of the organism. All cells, save the germ cells, are somatic cells.

The reversion of somatic cells to iPS cells provides a source of pluripotent stem cells without the need for human preimplantation embryos while providing the properties of human ES cells which make them useful for, inter alia, therapeutic applications such as treatment of juvenile diabetes and spinal cord injury. Current methods for generating iPS cells, however, employ retroviral vector delivery systems (e.g. lentiviral vectors) to deliver the necessary genes to mammalian cells. These methods are undesirable due in part to the limited payload size and the tendency to incorporate viral sequences into the eukaryotic host genome in random locations at high frequency. Moreover, induction of iPS cells from human somatic cells requires a high transduction frequency. In order to achieve high transduction frequency, Takahashi introduced the mouse receptor for retroviruses into adult human fibroblast target cells and observed a transduction efficiency of 60%.

Bactofection methods of the present invention allow transfection of eukaryotic host cells with virtually unlimited size constraints, without modification of the host cell chromosome and with surprisingly high efficiency. Thus, in one aspect, the present invention is directed to a method for introducing and expressing nucleic acid or gene (e.g. encoding a transcription factor) in a mammalian cell comprising infecting the mammalian cell with an invasive bacterium comprising a eukaryotic expression cassette, said expression cassette comprising said gene and said bacterium having a clean genome, wherein the bactofection efficiency is greater than about 1%, greater than about 5%, greater than about 10%, greater than about 20%, greater than about 30%, greater than about 40%, greater than about 50%, greater than about 60%, greater than about 70%, greater than about 80%, greater than about 90%, greater than about 95%, greater than about 96%, greater than about 97%, greater than about 98%, greater than about 99% or anywhere therebetween. Preferably the bactofection efficiency is greater than about 90%.

In one embodiment, the present invention provides a method for producing an iPS cell from a mammalian somatic cell comprising infecting the mammalian somatic cell with an invasive reduced genome bacterium comprising one or more vectors comprising one or more eukaryotic expression cassettes, said one or more expression cassettes comprising genes encoding at least Oct3/4 and a member of the SRY-related HMG-box (Sox) family of transcription factors selected from the group consisting of Sox1, Sox2, Sox3 and Sox1S. Preferably, the Sox factor is Sox2. The one or more eukaryotic expression cassettes preferably further comprise gene(s) encoding one or more transcription factors selected from the group consisting of: NANOG; LIN28; and a member of the Kruppel-like factors (Klfs) family of transcription factors. Preferably, the Klf factor is selected from Klf1, Klf2, Klf4 and KlfS. More preferably, the Klf factor is selected from Klf2 and Klf4. Most preferably, the Klf factor is KIlf4. Genes encoding transcription factors may be delivered to the somatic cell singly (i.e. sequentially) or may be delivered in combination

The generation of iPS cells from somatic cell precursors may be confirmed by, inter alia: embryonic stem (ES) cell morphology; expression of cell surface markers including, without limitation, SSE-1(−), SSEA-3(+), SSEA-4(+), TRA-1-60(+), and TRA-1-81(+); gene expression pattern characteristic of ES cells; expression of telomerase activity; and the capacity to differentiate into multiple lineages.

Plasmids useful in bactofection methods of delivering genes (e.g. those encoding transcription factors) to somatic cells comprise at least one eukaryotic expression cassette capable of expressing the gene in eukaryotes. Multiple eukaryotic expression cassettes may be delivered that express any combination of genes encoding, e.g. all or parts or any combination of transcription factors. The plasmids may also comprise a prokaryotic expression cassette comprising a gene encoding an invasive or invasion protein such as the invA gene of Yersinia pseudotuberculosis so that the clean genome bacteria acquires an invasive phenotype.

### Deletion Methodology

Methods for deleting DNA from a bacterium such as E. coli are described in U.S. patent application Ser. No. 10/057,582, U.S. Provisional Application Ser. No. 60/409,080 and PCT/US03/01800, all of which are herein incorporated by reference in their entirety. Tables 1, 3, 7 and 8 below describe exemplary deletions. Preferably the deletion methods resulting scarless deletion which avoid potential sites for recombination and thus genome instability. Table 6 depicts growth characteristics of certain MDS strains.

### EXAMPLE 1

### Transformation Frequency of MDS Clean Genome Bacteria

Exogenous DNAs are typically in the form of self-replicating plasmids. It is often desirable to incorporate DNA encoding plasmid maintenance functions into the genome of E. coli deletion strains in such a way that host bacterial cells will maintain the plasmid DNA as they divide and grow. The process of exogenous DNA introduction into bacterial host is called transformation and organisms that harbor exogenous DNA are called transformed organisms. There is a need in the art for E. coli strains with high transformation efficiency.

E. coli strain MDS39 was constructed by making 39 deletions (approximately 14.1% of the genome) in parental E. coli strain MG1655 and was found to be efficiently transformed by electroporation. This high efficiency of transformation extended to intake of a large size BAC (Bacterial Artificial Chromosome) DNA, which makes the strain MDS39 particularly valuable for the wide range of applications.

E. coli strain MDS41 was made from MDS40 strain by deleting the tonA gene using methods described above.

The multi-deletion E. coli strain MDS43 derived from sequenced E. coli K-12 was developed from K-12 strain MG 1655 which is non-pathogenic; the MG 1655 genome was sequenced and all the deletion junctions in MDS43 have been sequenced; furthermore, the MDS genome can be easily and economically resequenced by chip technology, permitting the system to be completely defined, and giving an unprecedented level of assurance that the vaccine contains no hidden threats. Most cryptic or potential pathogenic genetic elements have been removed. All IS and phage elements have been removed as well and no mechanisms of outward horizontal transfer remain, and a planned modification will prevent DNA uptake from the environment. Plasmids and antibiotic resistance markers may be eliminated by insertion into the stable genome before the clinical phase. K-12 strains are normal constituents of gut flora and MDS43 contains only those genes that are required for vaccine efficacy.

Starting from the sequenced K-12 strain MG1655, rationally designed deletions have removed phage receptors, intracellular, periplasmic and membrane proteinases, all recombinogenic or potentially mobile sequences, and horizontally transferred segments. The techniques involve selection for homologous recombination in vivo, such that even large (100 kb) segments of the E. coli genome can be deleted, modified or replaced. Others improved the controllability and efficiency of recombination.

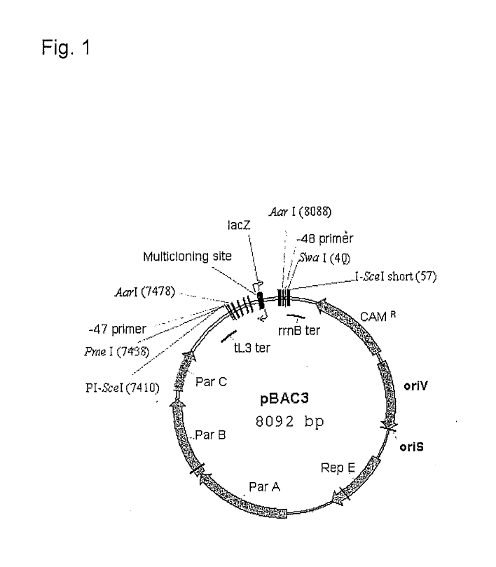

Maps of the deletions made in K-12 to produce MDS43 are shown in FIG. 1 of PCT/US03/08100.

To test the transformation efficiency of E. coli strain MDS39 in harboring and stably maintaining exogenous DNA, three strains: DH10B, MDS31 and MDS39 were grown under standard growth conditions to optical density of 0.5 at 600 nm. Cell cultures were spun down, cell pellets were washed several times with water and finally resuspended in water (at 1/1000 of the original culture volume). 25 ng of either pBR322 DNA or methylated BAC DNA or unmethylated BAC DNA was added to 100 μl of the cell suspension and subjected to electroporation using standard electroporation protocol, e.g., 1.8 kV and resistance of 150 ohms in a 0.1 cm electroporation cuvette using an Invitrogen Electroporator II™ device. BAC DNA methylated at the EcoK sites and pBR322 DNA were prepared in E. coli strain MG1655 using standard protocols. Unmethylated BAC DNA was prepared in E. coli strain DH10B.

Tables 3 and 5 show that both strains, MDS31, and MDS39, and MDS40, are efficiently transformed by pBR322 DNA with molecular weight of 4,363 base pairs and by methylated BAC DNA with molecular weight of 100,000 base pairs. The efficiencies of transformation with methylated BAC DNA for strains MDS31 and MDS39 are comparable with the efficiency of transformation for strain DH10B which is currently regarded as one of the strains with the best transformation efficiency.

When transformed with unmethylated BAC DNA, the efficiency of transformation for strain MDS39 was higher than the efficiency of transformation for strain DH10B (Table 3), while the efficiency of transformation for strain MDS31 was lower than the efficiencies of transformation for both strains MDS39 and DH10B. The low efficiency of transformation for strain MDS31 is due to the fact that the unmethylated DNA is a subject to restriction in the strain because MDS31 is a r⁺m⁺ strain, while both strains DH10B and MDS39 are r⁻m⁻ strains.

Recent work with MDS39 revealed the possible presence of a residual insertion sequence IS5 in sequence gb_ba:ecu 95365. In order to determine the effect of deleting of deleting the resident IS sequence from MDS39, procedures described herein were used to delete the sequence. The endpoints of the deletions in MDS40 are strains in Tables 8 and 9. The resulting strain MDS40 was then tested for its transformation offering and growth characteristics (Results) as discussed below.

Electroporation-competent cells were prepared as described in the Invitrogen Electroporator II Manual. Briefly, a 200-ml culture was grown to OD₅₅₀=0.5, then cells were harvested by centrifugation and washed twice in ice-cold water and once in ice-cold 10% glycerol by repeated centrifugation and suspension. At the final step the cell pellet was suspended in 0.4 ml 10% glycerol, aliquoted in 40 μl portions and stored at −80° C.

The cells were typically electroporated with 10-100 ng quantities of plasmid DNA at 1.8 kV and a resistance of 150Ω in a 0.1-cm electroporation cuvette using the Electroporator II device (Invitrogen). Cells were then diluted with 1 ml LB, incubated in a shaker for 1 h, and plated on selective medium.

Several experiments were done, results may vary by an order of magnitude. The average of 2 typical, independent experiments (2 parallels each) are shown in Table 5.

To determine transformation efficiencies for MG1655, MDS40 and DH10B, chemical transformation methods were also used. Competent cells were prepared by a simple method. A 50-ml culture was chilled and harvested by centrifugation at OD₅₅₀0.4, then washed twice with 1/20 volume of ice-cold CaCl₂ solution (10 mM Tris pH 7.5, 15% glycerol, 60 mM CaCl2) with repeated centrifugation and suspension. Cells were then incubated on ice for 1 h, aliquoted in 200-μl portions and stored at −80° C.

For transformation, cells were typically mixed with 100 ng plasmid DNA, incubated on ice for 30 min, heat-shocked at 42° C. for 2 min, then 0.8 ml LB was added. Cells were incubated at 37° C. for 0.5-1 h, then dilutions were plated on selective medium. Results are shown in Table 6.

### EXAMPLE 2

### Constructing Eukaryotic Reporter Plasmid LacZ with an Intron

To provide a test of correct transcript processing in target cells, a modified lacZ gene was introduced into a gWiz plasmid (Gene Therapy Systems) downstream of a CMV promoter. The lacZ gene was engineered to resemble a eukaryotic gene by insertion of an intron. The Human β-globin second intron was amplified by PCR from a genomic clone of the entire human globin locus, using primers designed to correspond precisely to the intron ends. The PCR polymerase used was PfuUltra, a very high fidelity enzyme leaving blunt ends. The agarose gel-purified product was ligated into an Eco47III site in the lacZ gene, 1919 by from the start of the 3144 by gene. E. coli DH10B transformed by the resulting plasmid grew as white colonies on IPTG/Xgal agar indicating no synthesis of active β-galactosidase, whereas the parent was blue. The intron and junctions were sequenced to confirmation of the structure.

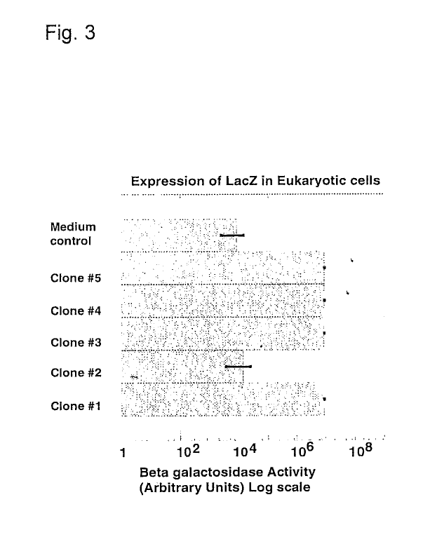

Transient transfection into mammalian cells was performed with candidate plasmids, and transfectants were assayed for β-galactosidase. Accurate intron splicing was demonstrated in 293T cells that were transfected with 2 ug each of 5 independent clones of the plasmid using Fugene non-liposomal transfection reagent (Fugent, LLC). Activity was measured using a fluorescent substrate for β-galactosidase and the responses were read on an automated plate reader and expressed in arbitrary units of fluorescence. The resulting data are shown in FIG. 3. The cells exposed to the transfection agent alone produced approximately 10⁴ units of fluorescence. By contrast, transfectant clones 1,3,4 and 5 elicited approximately 1000-fold higher responses. Clone 2 was no more active than the medium control. On sequencing, this clone was shown to have a single base deletion at one of the splice junctions. These results taken together provide strong evidence that the constructs are expressed only in eukaryotic cells, presumably by RNA splicing as expected.

The gWIZ-LacZ reporter was then tested in bactofection experiments with Shigella flexneri 2a vaccine strains CVD1203 (Kotloff et al., 1996 Infect Immun 64:4542-4548) and CVD1208 (Pasetti et al., 2003 J. Virol. 77: 5209-5217). Each of the strains was transformed either with beta-galactosidase expressing gWIZ-LacZ reporter (intron expression +) or with non-expressing negative construct gWIZ-LacZ (intron expression). Once the plasmids were introduced into the respective Shigella strains, the strains were checked for Congo red, and IpaB expression. Colonies positive for both were selected for bactofection experiments. HeLa cells (5×10⁴ per well) were incubated for 2 h with a late log phase culture of the appropriate bacteria at a MOI of 5:1. After 2 h, bactofected cells were rinsed 5× with media containing 100 ug/ml Gentamicin and then incubated overnight in the same media. At 21 h the cells were fixed and then stained with X-gal to visualize β-gal expression. The data shows that expression of gWIZ-LacZ reporter was detected in bactofection experiments with both CVD1203 and CVD1208 strains.

It is expected that the clean invasion plasmid will function in all of the deletion strain including MDS39, MDS41, and MDS43 and cultured mammalian cells with at least the same efficiency in the invasion assay as the native Shigella plasmid indicating that no other Shigella or E. coli genes are necessary for host cell entry and DNA delivery at least in vitro. Expression of the reporter lacZ gene will confirm that the plasmid DNA is being delivered into the target cells. This report can monitor delivery by any mechanism.

Human monocyte-derived dendritic cells (MDDC) are derived from highly purified populations of peripheral blood monocytes by culturing in the presence of IL-4 and GM-CSF. MDDC derived using these methods express classic markers of this subset and can be differentiated into functional mature dendritic cells by diverse agonists such as bacterial toxins. MDDC are capable of initiating primary immune responses in vitro when cultured with antigen and highly purified naive human T cells (see below).

The expression of the reporter gene is quantified in MDDC. Briefly, MDDC are electroporated using a commercial “Nucleofector” system (Amaxa, Gaithersburg, Md.). Transfection efficiencies in these experiments are typically of 15% to 25%. This system provides a positive control for bactofection studies.

Bactofection is quantified using MDDC harvested on days 5 or 6 after culture initiation by co-culture with varying multiplicities of infection (moi) of MDS strains carrying the LacZ reporter gene or control MDS strains lacking the LacZ reporter. The moi ranges from 0.001 to 100. The MDDC and bacteria are co-cultured for 24 hours and expression determined by flow cytometry at 24, 48, and 72 hours using a fluorogenic substrate as described. Optimal bactofection is defined as that moi that yields the highest frequency of positive cells as compared to the negative control (i.e., MDS strains that do not carry an expressible LacZ gene). The Amaxa system serves as a positive control. If GFP is used as the reporter (in order to use LacZ+ MDS strains (see above)), fluorescence intensity is read out directly on the flow cytometer without having to use an exogenous substrate. Besides GFP, yellow fluorescent protein (YFP), and red fluorescent protein (RFP) can also be used as reporters.

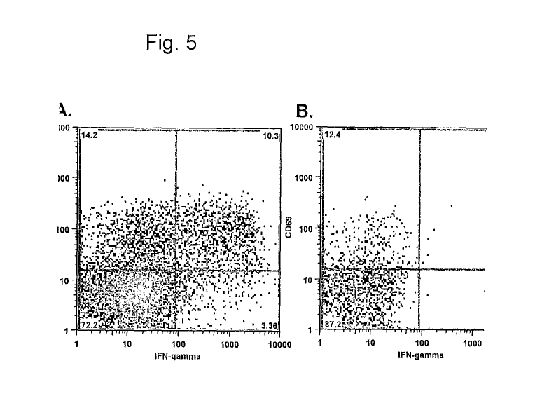

The primary immune response can be quantified by the extent of cell division and, in addition, by changes in the frequencies of activation/memory T cell subsets defined by surface markers and effector functions defined by cytokine/chemokine secretion. Furthermore, the system works equally well for nominal antigens, such as hemocyanin or bacterial proteins, superantigens, and alloantigens where the principal difference among these responses is quantitative and inversely proportional to the precursor frequency (ms in preparation). These changes occur over the first week of activation, permitting the rapid assessment of a primary response. Most important, between the second and third week of culture, the cultures are dominated by a population of small lymphocytes that have divided (as determined by down regulation of Carboxyfluorescein-succimidyl-ester (CFSE)) and this population contains memory-effector cells that are capable of a secondary response when co-cultured with autologous MDDC and antigen. The results of this analysis are shown in FIG. 5.

In this analysis, normal MDDC and highly purified naive CD4+ T cells were cultured for two weeks as described in except that 10 ug/ml of a total protein extract of Salmonella typhi, Strain Ty21a, was used as the immunogen. Fourteen days after the initiation of the cultures, the cells were harvested, washed, and cultured for 6 hours in the presence of MDDC or MDDC plus 10 ug/ml of the immunogen. Cytokine secreting cells were determined after a 6 hour incubation using CD69 as an acute activation marker (y axis) and IFN-γ as the cytokine (x-axis).

As shown in FIG. 5, a potent antigen-specific response was elicited as judged by the high frequency of CD69+ IFN-γ+ cells in panel A (10.3% of the total) as compared to panel B (0.17% of the total). The initial gating was carried out on small resting cells that had divided as determined by forward light scatter, orthogonal light scatter, and USE down regulation. Responses were not observed when the immunogen was excluded from the initial culture (data not shown). This system is highly quantitative and data can be obtained and analyzed in approximately three-weeks.

Once an optimal moi for MDDC has been determined for a particular MDS LacZ combination, the bactofected MDDC can be used to initiate a primary immune response by co-culturing with autologous naive CD4+ T cells. Since the immunogen is a complicated mixture of E. coli antigens as well as the lacZ DNA vaccine it is important to determine whether the LacZ was immunogenic using the short-term secondary response system described above. This may be done by bactofecting MDDC with the optimal moi of a MDS LacZ strain and co-culturing with autologous naive CD4+ T cells for 14 days. On day 14, the cultures are harvested and restimulated for 6 hours with freshly isolated (day 5 or 6) autologous MDDC plus 20 ug/ml of purified lacZ. Brefeldin-A is added after the first hour of stimulation to block the secretion of IFN-γ. After restimulation, the cultures are permeabilized and stained for CD69 and IFN-γ using standard procedures. Controls for the primary culture include cultures simulated with MDS with LacZ negative plasmids (negative), and cultures stimulated with 20 ug/ml of purified lacZ (positive control). Controls for the secondary culture include cultures stimulated with E. coli protein extracts (prepared by French press and ammonium sulfate precipitation (data not shown)) and cultures stimulated with medium alone.

### EXAMPLE 3

### Constructing Amplifiable BAC Plasmid Vector

The amplifiable pBAC3 can be maintained at a low copy number and induced to high copy number by turning on a second origin of replication. It serves at least two purposes in this project, first to provide a stable clone of the invasion locus from the Shigella virulence plasmid. Secondly, (at a later stage), the promoter that drives copy number amplification is replaced with one that is induced in the intracellular environment. The BAC can also be fitted with a prokaryotic or eukaryotic promoter to express the antigen protein from the cloned vaccine DNA. This vaccine DNA is amplified on entering cells of the immune system, and expression of antigen is maximized where it is most useful.

pBAC3 is a derivative of pBeloBAC11, a low copy number vector in which DNA fragments of at least 100 kb may be stably cloned. As can be seen in FIG. 1, the original replication system based on oriS maintains the copy number at 1-2 per cell. The addition of a second replication system from the broad host-range plasmid RK2, consisting of oriV and replication protein TrfA, allows the plasmid to amplify to ˜100 copies per cell upon induction, even with large inserts (Wild et al., 2002 Genome Res. 12:1434-1444). Control of the high copy system is exerted by the E. coli arabinose operon promoter araBAD and its transcriptional activator AraC, driving expression of trfA. The system is induced by arabinose but in its absence is completely inactive, giving tight control of trfA expression.

pBAC3 is shown in FIG. 1. Other features are LacZ blue/white screening for inserts, a multi-restriction site polylinker, several Type IIS (asymmetric) and other rare restriction sites. The cloning region is flanked by transcription terminators that prevent readthrough from plasmid promoters. Standard M13 sequencing primer sites are present at either end of the cloned insert. Chloramphenicol transacetylase (CAM) provides a selectable drug-resistance marker. Currently TrfA is supplied in trans by a separate plasmid, but the trfA gene may also be incorporated into pBAC3. The pBAC3 vector has no origin of transfer and no transfer or mobilization genes, and therefore cannot be mobilized into other bacteria in vivo.

### EXAMPLE 4

### Clean Genome E. Coli MDS41 Functions as a Vaccine Delivery Vehicle

This example teaches that the clean genome E. coli MDS41, MDS42 and MDS43 may function as a DNA delivery vehicles in vitro by using conditions and cell lines already demonstrated suitable for “bactofection” (delivery of DNA from bacteria into mammalian cultured cells). Such cells include but are not limited to cell lines including but not limited to ATCC Nos. CCL62, CCL159, HTB151, HTB22, CCL2, CRL8155, HTB61 and HTB104.

To assess the potential of E. coli MDS41 strain as a delivery vehicle in vivo for DNA vaccines, the strain is transformed with the lacZ reporter plasmid, from which beta-galactosidase is expressed in eukaryotic cells only when the transcript undergoes correct splicing. The effectiveness of the clean invasion plasmid in enabling MDS41 to enter the target cells is compared with the native Shigella virulence plasmid in an invasion assay. Bactofection is assayed with both invasion and reporter plasmids present in MDS41. Positive controls include direct transfection of the plasmid using Fugene and bactofection of the plasmid using Shigella flexneria strain 15D that is commonly used for bactofection studies (Sizemore et al. Science, 270: 299-302 (1995)). Negative controls include the plasmid vector without an intron delivered as both naked DNA using Fugene and as a Shigella-delivered DNA using strain SL7207 (Fouts et al. Vaccine 13: 1697-1705 (1995)).

The initial conditions already established for 15D will be used. Briefly, 293T cells (Dubridge et al. Mol. Cel. Biol. 7: 379-387 (1987)) will be grown to late log phase and exposed to bacteria grown under conditions that render them maximally invasive. Invasion is determined using the gentamicin resistance assay as described (Elsinghorst Methods Enzymol. 236: 405-420 (1994)). Bactofection is quantified using the fluorogenic beta-galactosidase substrate fluorescein di-beta-galactopyranoside and an automated plate reader (Victor, Perkin-Elmer). The responses are standardized using micrograms of total cell protein determined by Coomassie Blue binding, as the denominator. The multiplicities of infection are ranged from 0.01 to 100 in ½ log intervals. Expression is determined over a 72 hour period by sampling triplicate cultures every 24 hours.

Vaccine delivery can be improved by increasing the copy number of either the prokaryotic expression cassette to enhance the production of the soluble mStx2 protein or the eukaryotic expression cassette contained on the DNA vaccine in the MDS strain. pBAC3, an amplifiable BAC vector that normally persists as a low copy number plasmid but that can be amplified at least 100-fold by a second replication origin, oriV, operated by a inducible mutant replication protein TrfA203 can be used to accomplish this purpose. Wagner et al., Mol. Microbiol. 44(4):957-70 (2002), found that increased copy number of phage genomes was the “most quantitatively important mechanism” of Stx1 production and could play a similar role to enhance the immunogenicity of the delivered mStx2.

To create an invasive MDS strain, the invA gene from Yersinia pseudotuberculosis is cloned onto single copy plasmid, pBAC3, to create pBAC3-invA. The invA gene is selected because introduction of this single gene confers invasive phenotype onto otherwise non-invasive E. coli strains. MDS42 and MDS43 were then transformed with (pBAC3-invA) and their resulting invasive capacity assessed in a gentamicin protection assay. CaCo2 or HeLa cells were infected with different MOIs of bacteria, then, after 2 hours, washed thoroughly and treated with gentamicin to kill all bacteria that have not invaded. After another two hours, the cells are washed, lysed and the CFUs were determined. The data indicated that introduction of the invA gene is sufficient to facilitate invasion of CaCo2 and HeLa cells by MDS42 and MDS43 demonstrating that no further engineering of the MDS genome is needed for invasion. Furthermore, invasion by both MDS42 and MDS43 expressing invA is as efficient as the invasion conferred by Salmonella typhi strain Ty2.

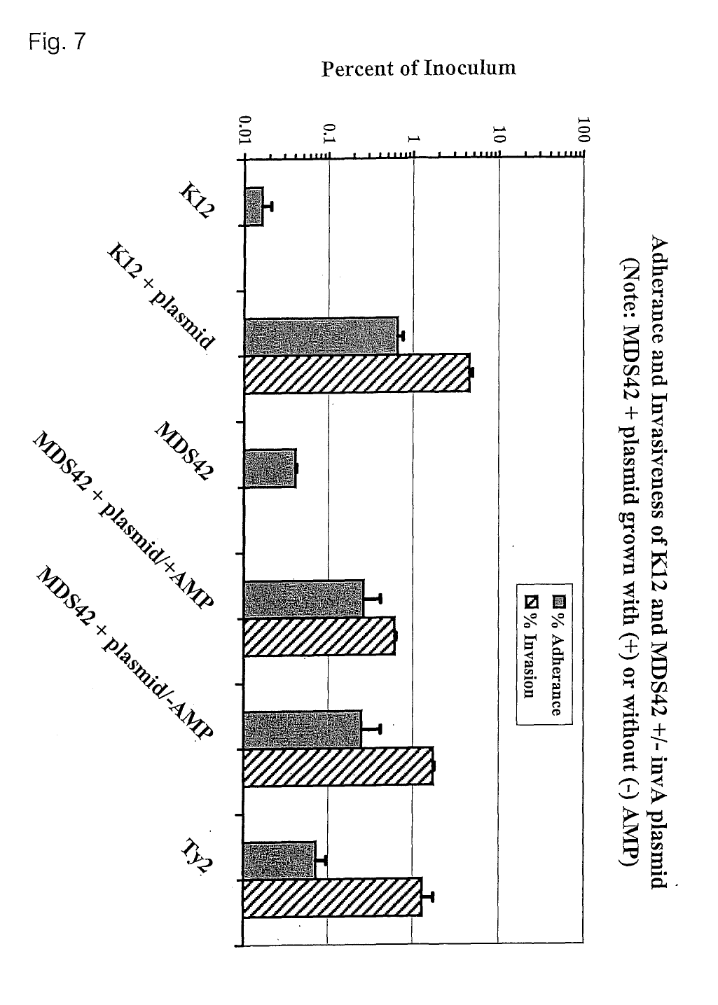

Experiments were also conducted to determine an adherence and invasiveness of K12 and MDS42+/−invA plasmid HeLa cells (5×10⁴ per well) were incubated for 2 h with a late log phase cultures of the appropriate bacteria at a MOI of 5:1. After 2 h the cells were rinsed 5× with media containing 100 ug/ml Gentamicin and then incubated overnight in the same medium. At 21 h the cells were fixed for 5 min and then “stained” with X-gal as per manufacturers protocols. When infecting bacteria deliver a reporter plasmid encoding the lacZ gene, beta-galactosidase expression from lacZ on the plasmid produces a blue product from the chromogenic substrate. Colored Hela cells may be counted by microscopic observation or automatically by fluorescence-activated cell-sorting (FACS) if a fluorogenic substrate is used. Viable bacteria may also be recovered from washed HeLa cells on lysis with detergent. Data showing adherence and adhesiveness of E-coli, K12 and MDS42 with and without the invA plasmid as shown in FIG. 7.

### EXAMPLE 5

### Constructing an Invasion Plasmid of Shigella's Invasion Locus

Invasion capability can be supplied by any mechanism employed by invasive bacteria, like that of Yersinia and Listeria (single “invasin” or “internalin” protein), or Shigella and Salmonella (multiple effectors dependent on type III secretion to deliver the signal triggering uptake of the bacteria into the target cell). Invasion mechanisms recently reviewed in Cossart, P., and P. J. Sansonetti 2004. Science 304:242-248 are not fully understood. Essentially, bacterial invasion proteins gain access to the interior of the target cell and subvert host signaling systems to reorganize the cytoskeleton and bring about engulfing of the bacterium. Other mechanisms exist, used by microbes and parasites (Sibley, L. D. 2004 Science 304:248-253).

For full pathogenicity of Shigella in vivo, genes in various pathogenicity islands in the Shigella chromosome are required but the virulence plasmid itself was sufficient to enable E. coli K-12 to invade cultured cells, providing proof of principle (see, e.g., Grillot-Courvalin et al., Cellular Microbiology (2002) 4(3), 1776-186; Cicin-Sain et al., J. Virol. (2003) 8249-8255; Narayan et al., N. Acct. Res. (2003) 31: and Pilgrim et al., (2003) 10:2036-2045). The objective of this example is to isolate the invasion (ipa-mxi-spa) locus away from the large number of IS elements, which comprise >50% of this invasion plasmid. Shigella was initially chosen as the source of these genes because macrophage apoptosis is slower than that caused by Salmonella, allowing more time for antigen expression and processing. Not all of the components of the bacterial invasion function are fully characterized and some genes encoded within the invasion locus appear to be dispensible for invasion in vitro. Some genes in the locus are regulated by the activity of the secretion system. A gene required for lateral spread of bacteria from cell to cell within the epithelium, icsA is encoded on the native plasmid but outside the invasion locus and, if not required for efficient antigen delivery, will be excluded to limit persistence and attenuate the consequences of infection.

Several approaches are possible. The best choice is a PCR-based strategy which is clean and offers greatest flexibility for engineering. No intermediate subcloning of segments containing IS elements is involved, therefore no instability should be encountered.

The Shigella virulence plasmid invasion locus can be divided into three segments of 11 kb, 13 kb and 6 kb comprising the main operons. High-fidelity polymerases are available (PfuUltra from Stratagene and Platinum Pfx from Invitrogen) that now function with an error rate of about 1-2×10⁻⁶ in amplified DNA, thus can faithfully amplify at least 10 kb. Based on our previous experience with long-PCR, these are realistic amplimer sizes to obtain, especially now that highly efficient polymerase mixes are available. Purified virulence plasmid DNA is available as template, so the number of cycles required for amplification can be limited, further guarding against polymerase errors and PCR artifacts. Using one of these enzymes we will PCR amplify the three constituent operons separately. The operon junctions need to be reproduced carefully since the promoters apparently overlap into upstream genes. The gaps between gene ends at the borders of the PCR fragments are only 14 bp and 4 bp long. The primers will contain sequences incorporated into the amplimers to allow correctly oriented ligation, for example via non-palindromic restriction sites, allowing directional cloning into the pBAC3 vector. If necessary to preserve transcription, the linker sequences will then be deleted in vivo to achieve precise joining of the three segments using oligo-templated recombination. Other PCR strategies are possible, e.g., overlap extension or chain-reaction cloning.

Alternatively, the locus could be cloned by conventional restriction fragment isolation, though not in a single piece. A large (29 kb) fragment with BamHI and XhoI ends, and an adjacent small (1.8 kb) fragment with BamHI ends covers the entire ipa-mxi-spa region including the positive regulator virB. Agarose gel-purified restriction fragments would be ligated into pBAC3 using an oligo linker/adapter to convert the XhoI end to fit the unique PmeI site in the vector. The small fragment may then be added at the BamHI and PCR used to screen recombinants for the correct orientation of the small fragment. This construct is clean of IS at the BamHI end, but has about 200 bp of IS600 at the XhoI end. This may need to be removed by targeted oligo-directed recombinational deletion.

The invasive phenotype may also be modified adding back certain plasmid genes from outside of the invasive locus. Candidates include five members of the ipaH gene family (function unknown but their gene products have intriguing similarities to mammalian receptor proteins) and the regulator virF. These could be readily added to the construct in pBAC3 by PCR-based technology.

The invasion locus can be transferred into the MDS41 chromosome where it will be passively replicated. Although small plasmids would not be expected to impose a metabolic burden on the bacterial host, the invasion locus cloned into pBAC3 would be a 38 kb plasmid which if induced to 100 copies per cell, would be a replication task approaching that of the genome. This would certainly place a replication and gene expression burden on the bacterium. With the invasion locus on the chromosome, the selective marker and vaccine DNA would comprise a much smaller construct, allowing maximal scope for adding combinations of vaccine DNAs. A eukaryotic promoter such as the CMV promoter can be added to pBAC3 to convert it into an expression vector for eukaryotic DNA.

The 30 gene ipa-mxi-spa region of the Shigella virulence plasmid encodes a type III secretion system and effectors whose activities are necessary for invasion of human cells. Since the natural plasmid is heavily loaded with IS elements that present a risk factor, a clean plasmid with the IS-free ipa-mxi-spa region cloned into pBAC3 is constructed to accomplish tasks of the instant invention.

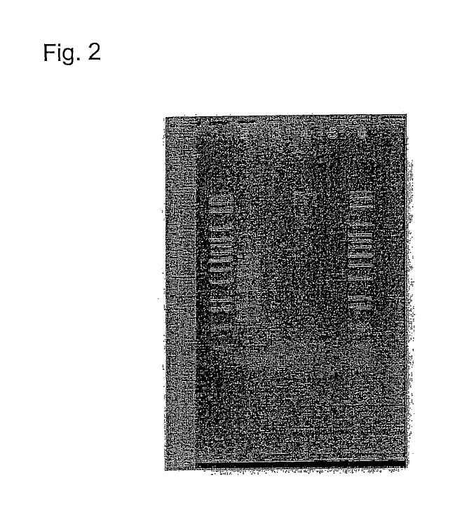

FIG. 2 shows successful amplification of 30 kb Shigella invasion locus. PCR was performed with a variety of high fidelity polymerases and conditions, using purified Shigella pINV plasmid DNA as template. Primers were designed at the ends of the region, avoiding the flanking IS elements. Most reactions gave no amplimer or multiple small amplimers, but one case was successful, giving a clean single band with a minimum of background. To resolve the PCR products, 0.5 SeaKem Gold agarose gel electrophoresis was used. In the figure, lanes 1 and 6 contain size markers, of which the top three bands are 10, 20 and 40 kb. Lane 2 shows PfuTurbo polymerase products; lanes 3 and 4 show products of Platinum Taq DNA polymerase High Fidelity at different Mg⁺⁺ concentrations, with the successful 30 kb band in lane 4. Lane 5 is a negative control. A total of 33 cycles were used in the successful reaction.

As an alternative to the Shigella virulence plasmid with the complexities of the invasion locus and its regulation, the inv gene from Y. pseudotuberculosis can be tested. Invasin, the inv gene product, is sufficient to confer invasiveness on E. coli K-12 strains. Invasin targets f31-integrins on human cell surfaces, inducing internalization of Inv+ bacteria by cultured non-phagocytic cells. The plasmid pR1203 containing a 4.5 kb BamHI fragment encoding inv and its promoter (20) was introduced into MG1655 (the sequenced wild type K-12 strain), DH10B (a popular plasmid host) and MDS42.

### EXAMPLE 6

### Engineering MDS41 to Make a Non-Antibiotic Selectable Marker

To make MDS41 dependent on a resident plasmid (selection for maintenance of the vaccine-DNA-containing plasmid), an essential gene or segment of the chromosome containing an essential gene can be deleted. To allow deletion we must first supply a copy of the essential gene for complementation. The region containing the target essential gene is amplified by high-fidelity PCR followed by cloning into pBAC3, initially with the chloramphenicol resistance (CAM) marker intact. The chromosomal target gene will then be deleted by targeted recombination. By targeting the chromosomal deletion endpoints outside the plasmid-encoded essential gene segment, the plasmid gene will not be removed. Finally, the CAM marker is removed by the same technique.

For a strong selection without adverse effects, we will use an essential gene that is absolutely and continuously required, for example, a gene whose product is involved in information transmission. Suitable candidates include the general replication enzyme DNA polymerase III (gene polC), tRNA synthetase genes thrS and ileS. Considering polC, there is no evidence that it can be replaced or complemented by a polymerase from any other species, so as a selection is most unlikely to be lost due to a horizontal transfer event. Other candidates of a different functional category could be used. For example, conditional mutants of two enzymes involved in synthesis of cell surface components that show rapid cessation of growth when non-permissive conditions are applied; murA (UDP-N-glucosamine-carboxyvinyltransferase; catalyzing the first step in murein biosynthesis) and lpxC (UDP-3-O-acyl N-acetylglucosamine deacetylase; an enzyme of lipid A biosynthesis). Several candidate genes can easily be processed at once, and tested for stable and reproducible physiology.

After the deletion of the chloramphenicol resistance marker on pBAC3 by the same strategy, the growth rate of MDS41/pBAC3-with the essential gene will be compared with that of MDS41 without the plasmid or deletion. Persistence of the BAC will be also be assayed by comparing numbers of viable cells at different stages along the growth curve and by quantitative PCR of a plasmid target other than the essential gene, from a fixed number of cells, also at stages along the growth curve. For the cell surface enzyme markers, the cultures will also be inspected microscopically for any changes in morphology.

### EXAMPLE 7

### A Single Chain Polypeptide Complex Containing the HIV-1 Envelope Glycoprotein and a CD4 Receptor Mimetic Peptide Elicits Broadly Cross-Reactive Neutralizing Antibodies Against HIV

The structure of the HIV gp120 envelope glycoprotein that is induced by its CD4 receptor is a potential model for the development of HIV vaccines that elicit neutralizing antibody responses. It was previously shown that cross linked complexes of HIV gp120 and soluble CD4 elicited cross-reactive antibody responses that neutralized primary HIV isolates irrespective of genetic subtype (Fouts, et al., 2002, PNAS 99: 118427). These neutralizing antibodies bound to a chimeric single chain complex (SCBaL/M9) that used the CD4M9 mimetic miniprotein sequence (Vita et al., 1999, PNAS 96: 13091-6) instead of CD4 to produce a constrained envelope structure. Two protease-stabilized variants of SCBaL/M9 elicit humoral responses in rabbits that neutralize a broad range of primary HIV-1 isolates across assay formats. Thus, SCBaL/M9 antigens may warrant further consideration as a vaccine component for eliciting humoral immunity against HIV. Such a vaccine component may be utilized.

### Neutralization of HIV-1 Isolates by Sera from Rabbits Inoculated with BaLgp120-CD4M9 Complexes

Sera from rabbits inoculated with the indicated immunogens were tested in two standardized neutralization assay formats. Naive sera collected from unimmunized animals were tested as controls. The HIV_{IIIB} is a T cell line adapted virus and is indicated as TCLA. All of the other viruses shown were passaged and titered only in primary human PBMC and were designated primary isolates. The values in Table 10 represent the reciprocal of the highest final serum dilutions interpolated from the dose response curves as inhibiting 50% (ID₅₀) of viral growth relative to control assays. Averages of triplicate or quadruplicate assays are shown.

### Neutralization Assays.

Format 1 (U373/CD4/coreceptor/MAGI). Immune and control sera filtered before use were tested in an assay system that uses U373/CD4/MAGI cells expressing either CCR5 or CXCR4 as targets.

Format 2 (PHA-stimulated PBMC). Sera were tested in assays with human peripheral blood mononuclear cells (PBMC) from HIV seronegative donors as targets. PBMCs were activated for 48 hrs with phytohemaglutinin and IL-2 prior to use. For either assay, IC₅₀ and IC₉₀ values were determined and are set out in Table 9.

SCBaL/mg antigens encoding DNA may thus be introduced into a eukaryotic expression cassette and introduced into a reduced genome bacterium, preferably E. coli to serve as a vaccine for inducing humoral immunity against HIV.

### EXAMPLE 8

### Stx2A Expression in the MDS Clean Genome Background

A DNA vaccine for Stx2A is constructed using the gWIZ vector (Gene Therapy Systems). The gWIZ vector consistently provides the highest levels of eukaryotic expression of any of the DNA vaccine vectors that are commercially available. This vector effectively delivers a reporter gene to HeLa cells. To optimize expression in human cells, the Stx2A gene is chemically synthesized using codons most frequently used in human cells. Eukaryotic expression of the resulting construct is confirmed by transfection of HEK 293 cells followed by immunoblotting using anti-Stx2A monoclonal antibody.

For bacterial expression the uhpT promoter is used. The optimized Stx2A gene is expressed in the bacterial periplasm on induction with glucose-1-phosphate. Variations of this example provide an opportunity to discover whether Shiga toxins are truly secreted by bacteria or are only released on bacterial lysis, and whether the internal transmembrane segment in A1 is important. Expression by either route from the resulting MDS43 strain is confirmed by immunoblot using anti-Stx2 monoclonal antibody.

Although the uhpT promoter is well suited to these test experiments, it is necessary to identify other invasion-inducible promoters so that the final strain does not carry duplicate sequences, which could promote recombination. To identify alternatives, gene expression of MDS43 invading human cells is tested by using Nimblegen DNA chips.

### EXAMPLE 9

### Marine Stx2 Toxicity

The murine protection model for Stx2 is a useful means to screen potential vaccine modalities against Stx2. This mouse model is simple, well-established, and widely used. In this model, CD-1 mice are challenged intraperitoneally with a lethal dose of purified Stx2 or culture supernatant from enterohemorrhagic E. coli strain O157:H7. Vaccine-mediated protection is monitored as the number of mice that survive for more than 72 hours after the challenge compared to unvaccinated controls. Protection in this model is strictly dependent on the presence of sufficient titers of neutralizing anti-Stx2 antibodies at the time of challenge.

To evaluate MDS42 based mStx2 vaccine candidates, an inoculum of 10¹⁰ CFUs of MDS42 vaccine strains is administered in PBS by oral gavage (feeding tube) or by intraperitoneal (IP) injection to mice that have been pretreated for 2 days with streptomycin (5 mg/ml in their drinking water). This approach depletes the normal commensal gut flora, reducing competition and facilitating colonization by introduced E. coli strains. A 48 hour treatment with streptomycin is sufficient to eliminate the commensal flora. After the inoculation, mice are returned to streptomycin treatment to prevent return of the commensal flora.

To prevent elimination of the MDS42 vaccine strains, streptomycin-resistant colonies are isolated prior to inoculation by passage onto Luria-Bertani plates containing 30-100 μg/ml streptomycin. Spontaneous mutations in ribosomal proteins that confer streptomycin resistance on E. coli are easily obtained and alleles that have normal growth rates are most unlikely to have unwanted side effects.

The longitudinal profile of the immune response over a 4-6 week period after inoculation is measured in order to establish an optimal immunization protocol. The resulting immune response may be assessed using a Stx2-based ELISA and neutralization of Stx2 activity in a Vero cell cytotoxicity assay. ELISA assays consist of serial dilutions of murine serum added to purified Stx2 adsorbed to plastic. Bound antibody are detected with horseradish peroxidase-labeled anti-mouse IgG. For Stx2 neutralization assays, serial dilutions of purified Stx2 will be mixed with serum (or vice versa) then added to Vero cell cultures. Western blots may also be used. Toxicity is assessed according to standard protocols. Additional immunizations may be performed to discern whether boosting improves the resulting immune response. The optimal protocol is defined as the immunization strategy that generates the peak humoral response 2-4 weeks post inoculation that is not enhanced by subsequent boosts.

After these initial time course experiments, challenge experiments are performed using the immunization protocol that generates the optimal antibody response. At the peak of the immune response, all groups are challenged with B2F1 supernatant containing wild type Stx2. This supernatant is titrated to define the minimum dose required to induce 100% mortality in the untreated animals. Grouped survival data is analyzed by the Fisher exact test with significant protection having a p<0.05 degree of survival compared to untreated controls. 10 animals/group are used to provide sufficient power (95%) to detect significant protection in only 20% of the animals.

Preliminary experiments have demonstrated that IP-injected mStx2 vaccines can be very effective in protecting mice against a lethal challenge of Shiga toxin. These experiments have also demonstrated that oral gavage-delivered mStx2 vaccines can protect mice against the lethal challenge of Shiga toxin but less effectively than when IP-injected. In these experiments, 6-8 week old female Balb/c mice were inoculated with MDS42 reduced genome bacteria carrying a plasmid with a mutant Stx2A (mStx2A) under the control of a CMV promoter. These mice were subsequently challenged with the lowest dose of Shiga toxin predicted to kill untreated mice. The mStx2A was created by starting with the gene from enterohemorrhagic E. coli (EHEC) O157:H7 strain EDL933 and generating two mutations on opposite sides of the active site pocket which eliminate the protein's toxic glycosylase activity without affecting its immunogenicity.

### EXAMPLE 10

### Design of Stx Mutants and Selection of Non-Toxic Mutants

To begin with, an active site deletion mutant (shown to be non-toxic) of the gene encoding the Stx2-A1 subunit was designed to lack a signal sequence so that the expressed polypeptide will remain in the bacterial cytoplasm. E. coli ribosomes are susceptible to Stx toxicity, so if the N-glycosylase activity remains in any of the mutant candidates, the ribosomes of the E. coli host will be inactivated. FIG. 6 shows residues identified as key components of the active site.

As a control, wild type Stx2-A1 is amplified by PCR without signal sequence, and to validate the selection method, is cloned into a plasmid with tight expression control by the T7 promoter, with T7 polymerase under separate control of the E. coli rhamnose promoter and transcriptional activator RhaC, members of the araC/xylS regulator family.

This system maintains tight repression when glucose is present but is induced by rhamnose. The Stx2A mutant is cloned with the same promoter. After electroporation of the plasmid into MDS43, the bacteria are plated on +/−rhamnose inducer to express the mStx and only those cells harboring non-toxic mutants survive to form colonies.

Once the selection system is validated, several mStx genes are constructed by PCR with mutations introduced in overlapping primers, using a synthesized codon-optimized StxA2 gene as the template. Genes with combinations of changes, in the active site and the Tyr residues that contact the adenine substrate are also created (FIG. 6).

The mutant sequence designs in the A1 fragment are analyzed by an antigenicity- or epitope-predicting computer program such as Lasergene Protean (FIG. 6), or more recently developed tools such as Conservatrix and Epimatrix. These latter programs search a submitted sequence for regions likely to bind MHC by comparison to a large database of known MHC-binding peptides. The results compared with the wild type sequence will show which mutations are likely to produce conformational changes that disrupt epitopes so as to avoid making any substitutions that significantly distort the structure. Epitope analysis has made a large impact on high-throughput methods to find vaccine candidates, reducing the number of candidates to be tested by several orders of magnitude.

Many mutant designs can be screened computationally and by the bacterial toxicity selection. Non-toxic clones will also be tested in a Vero cell assay until it is clear that the bacterial selection gives equivalent results. Non-toxic mutants are screened for ability to produce maximum quantities of protein that is recognized by Stx2 mAb. If the DNA vaccine mode is selected, candidate mutant genes are transferred to the gWIZ plasmid and transfected into HEL 293 cells for expression testing. Mutant Stx protein are assayed by immunoblot. If subunit protein modality is selected, protein production induced by addition of rhamnose to the culture is assayed by immunoblot in a similar manner. A small number of candidates that express well and react with the Stx monoclonal antibody are defined for protection tests in mice.

Candidate mStx2 genes are introduced into MDS43 as either a prokaryotically expressed subunit protein or to be expressed eukaryotically from a DNA vaccine depending on the optimal modality. The resulting MDS43 strains are then screened for efficacy in the murine protection model. Control groups include untreated animals as well as MDS43 strains with mStx2 AA. Candidates that exhibit significantly heightened immune responses and efficacy (p<0.05) as compared to MDS43 mStx2 AA. If MDS43 mStx2 AA inoculated animals exhibit complete protection from challenge, dose finding studies are performed. Such studies with B2F1 supernatant containing wild type Stx2 define the minimum dose required to induce 100% mortality in the MDS43 mStx2 AA inoculated animals.

### EXAMPLE 11

### Ebola Virus

Ebola virus is difficult to investigate because of the lethality and lack of antiviral therapy. Animal models include mice, guinea pigs and non-human primates. Of these, monkeys are considered to be the best predictive model for human infections, and guinea pig infections more closely resemble the human disease than mice. In both rodents, however, the virus must be adapted by serial passages. Details of the viral pathogenic mechanisms and the immune response to Ebola infection in humans are still poorly understood. The viral targets are monocytes and macrophages of the immune system, liver cells, and endothelial cells of the blood vessels. It is likely that the envelope glycoprotein (GP) is responsible for disruption of the immune response and that it, and the inflammatory reaction it provokes, lead to destruction of the vascular endothelium and disseminated intravascular coagulopathy. The consequent internal bleeding and hypotension can be fatal. The virus replicates very rapidly and contaminates the blood and other body fluids. Transmission is usually by direct contact, but the possibility of aerosol dissemination in a bioattack is taken seriously. Studies based on individual genes have allowed safer work including vaccine development. Nabel, Sullivan et al at the NIH/NIAID Vaccine Research Center, have developed DNA vaccines based on plasmids or a non-replicating adenovirus vector encoding Ebola GP and NP (nucleoprotein) genes. This group have demonstrated that a prime boost strategy using three intramuscular injections of plasmid-GP over 4-8 weeks and a later injected boost of adenovirus-GP/NP confers strong protective immunity in mice and macaques. A faster but less effective immune response was elicited by a single injected dose of the adenoviral-GP/NP DNA. These vaccines went into human trials in November 2003.

Bactofection with MDS E. coli may deliver a better vaccine by targeting a massive amount of DNA to macrophages compared with that delivered by intramuscular injection of naked DNA. GP and NP genes are synthesized by using the published sequence for the Zaire subtype, strain Mayinga (GenBank AF086033) and codon optimization for translation in human cells. These genes are then cloned into pBAC3 with an intracellular-induced promoter and optimized invasion system. Initial testing is done in the MDDC immunogenicity assay described above, and trials in animal models (mouse and non-human primate) follow to ascertain safety and protective immunity.

### EXAMPLE 12

### Bactofection Efficiency

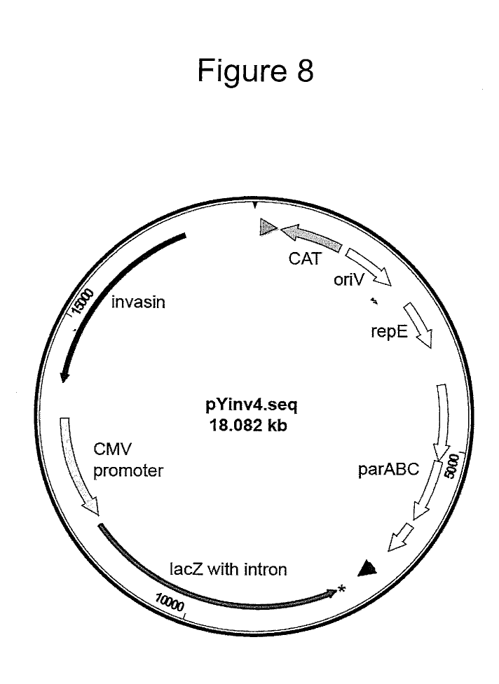

Vector pYinv4 is derived from plasmid pBAC16 and is shown in FIG. 8. pYinv4 comprises: (1) a first origin of replication, oriS, which allows the plasmid to be maintained as a single copy (2) a second origin of replication, oriV, which may be activated to high-copy number by expression of the trfA gene product (up to 100 copies/cell) (3) a CMV promoter controlling expression of a lacZ gene containing intron 2 from the human beta globin gene and (4) a Yersinia pseudotuberculosis invasion gene under its native promoter. Use of an intron in the lacZ gene minimizes expression in bacteria due to the “leaky” CMV promoter and confirms nuclear localization in the eukaryotic target cell. Invasin itself is not pathogenic but it enables E. coli to invade any mammalian cell type displaying the appropriate β1-integrin receptor subtypes, which are found on many tissues.

Vector pYinv4 was transformed into strain MDS42(recA)(ryhb)(trfA⁺). MDS42(recA)(ryhb)(trfA⁺) was constructed by deleting the recA and rhyb genes from MDS42, which lacks all transposable elements in order to avoid contamination of cloned DNA with these undesirable sequences. MDS42(recA)(ryhb)(trfA⁺) also contains the trfA gene under control of the chromosomal promoter for Ara_{BAD} to allow for plasmid copy number induction. No β-galactosidase activity was detected from the E. coli genomic lacZ gene.

The MDS42(recA)(ryhb)(trfA⁺) strain containing pYinv4 was grown in 0.02% glucose, and 0.2% arabinose and 12.5 μg/ml to induce trfA expression from the arabinose promoter and amplify plasmid copy number. The bacterial cells were grown overnight at 30° C. At an optical density (O.D.) of 3.3, the copy number induced cells were used either fresh or after freezing at −80° C. in 15% glycerol for bactofection of mammalian HeLa cells.

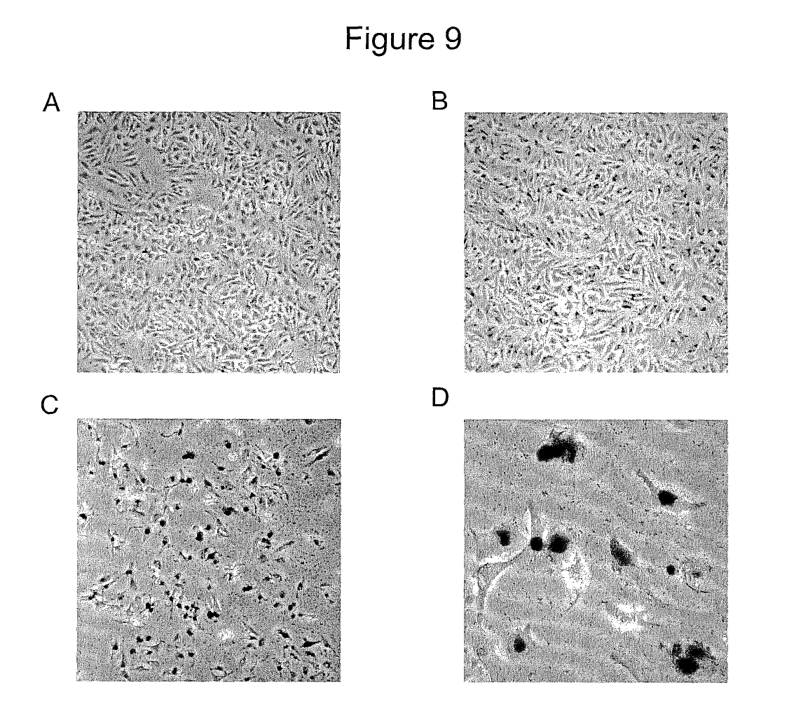

The fresh (FIG. 9, Panel B) or thawed (FIG. 9, Panels C & D) bacterial cells were added to mammalian HeLa cell cultures to a final multiplicity of infection of about 200 (5×10⁷ viable bacterial cells per 2.5×10⁵ viable HeLa cells) and allowed to infect for 2 hours at 37° C., 5% CO₂. Media (containing bacteria) was then aspirated and the HeLa cells were washed and then incubated with antibiotics (50 μg/ml gentamicin) overnight at 37° C., 5% CO₂. For colorimetric analysis, the HeLa cells were then fixed in 4% paraformaldehyde, rinsed, and incubated in β-galactosidase substrate solution and the percent of blue cells (measure of successful bactofection) determined. A bactofection efficiency of about 37% was observed for fresh bacteria (FIG. 9, Panel B). Surprisingly, the bactofection efficiency improved to about 99% when the transformed bacteria were frozen in glycerol prior to infection (FIG. 9, Panels C & D). The experiment was repeated multiple times with nearly identical results. Similar results were obtained with the following reduced genome strains: (1) MDS42(recA)(trfA⁺) and (2) MDS42(recA)(ryhb)(trfA⁺)(rpls⁺).

The above experiment was then replicated except that the plasmid was not induced (i.e., no arabinose was added). A bactofection efficiency of 0% was observed (FIG. 9, Panel A).

Bactofection efficiency was then measured in human embryonic kidney (HEK) 293 cells and in cultured murine cardiomyoctes using the procedure described above for bactofection of HeLa cells. Briefly, MDS42(recA)(ryhb)(trfA⁺) strain containing pYinv4 was grown in the presence of arabinose overnight, then frozen at 80° C. in 15% glycerol for bactofection of HEK 293 cells or cardiomyocytes. A bactofection efficiency of 75% was observed in HEK 293 cells and a bactofection efficiency of 45% was observed in cardiomyocytes. In contrast, when plasmid copy number induction was performed for only 2-3 hours (rather than overnight) and the transformed bacteria were not frozen in glycerol prior to infection, the bactofection efficiency dropped to 5-7% in HEK 293 cells and to 1-2% in cardiomyocytes. Similar results were also obtained in neonatal dermal human fibroblasts (HDFn).

Since MDS42(recA)(ryhb)(trfA⁺) contains endogenous lacZ (and therefore β-galactosidase activity), HeLa cells were bactofected with MDS42(recA)(ryhb)(trfA⁺) strain containing pYinv3, a vector identical to PYinv4 except that it does not contain the β-galactosidase insert, to control for the possibility that some of the observed blue cells resulted from bacterial lacZ expression. Very few to no blue cells were observed following colorimetric analysis of these HeLa cells, demonstrating that the high bactofection efficiency observed resulted from a eukaryotic splicing event.

### EXAMPLE 13

### Generation of iPS Cells from Somatic Cells

Genes encoding the Oct3/4 and Sox2 transcription factors and optionally one or more genes encoding the Nanog, Lin28, Klf1, Klf2, Klf4 and/or Klf5 transcription factors, are cloned into one or more eukaryotic expression cassettes of a suitable vector (e.g. pYinv4 with the lacZ gene replaced with the gene(s)). The eukaryotic expression cassette(s) containing each gene may be located on the same vector or on different vectors. Each eukaryotic expression cassette may comprise a single gene or multiple genes regulated by a single promoter, resulting in the expression of monocistronic or polycistronic mRNA, respectively.

Vectors comprising genes encoding the aforementioned transcription factors are used to transform an appropriate clean genome invasive bacterial strain (e.g. MDS42trfA⁺). Preferably, the vector comprises an inducible high-copy number origin of replication such as oriV, in which case the copy number of the vector is amplified to a very high copy number just prior to bactofection of the target mammalian cells. Preferably, the bacteria comprising the vectors are frozen at −80° C. in an aqueous glycerol solution (and subsequently thawed) prior to bactofection.

The live bacterial cells, comprising, separately or in combination, at least Oct3/4 and Sox2 and optionally one or more of Nanog, Lin28, Klf1, Klf2, Klf4 and/or Klf5 are then added to somatic mammalian cell cultures, preferably human mammalian cells, more preferably human fibroblasts, and allowed to infect for two hours. The mammalian cells are then washed with antibiotics, supplied with fresh media and cultured in vitro.

The cultured cells are monitored for the appearance of human embryonic stem (ES) cell-like morphology (compact colonies, high nucleus to cytoplasm ratios, prominent nucleoli). iPS colonies are expected to begin appearing at about day 12. Colonies with human ES cell morphology (iPS colonies) are picked. More detailed analysis may be performed on a subset of the iPS cells such as (1) testing for telomerase activity (2) testing for expression of human ES cell-specific cell surface antigens SSEA-3, SSEA-4, Tra-1-60 and Tra-1-81 (3) gene expression analysis (e.g. by microarray) and/or (4) ability to differentiate. iPS cells may be identified by morphology, expression of telomerase activity, expression of human ES cell-specific surface antigens, gene expression profile characteristic of human ES cells, and/or similar differentiation potential to human ES cells. The iPS cells may be treated like human ES cells for the purposes of culturing, etc.

### EXAMPLE 14

### Testing Safety-Enhancing Systems of the MDS Strains

A bacterial lysis cassette and a DNA restriction system were separately evaluated for their ability to enhance the safety of MDS strains compared to industrial and clinical research strains.

First, an inducible lysis system was evaluated that can be turned on following invasion in order to limit bacterial persistence and enhance payload release at the target site. To accomplish this, a segment from the E. coli bacteriophage lambda lysis region was cloned including the R and S genes as well as upstream sequences that regulate expression. The S gene encodes a “holin”, enabling the product of the R gene, a muramidase, to penetrate the cytoplasmic membrane and degrade the peptidoglycan layer resulting in bacterial lysis. This cassette was spliced to a T7 promoter in an expression plasmid which was then transformed into MDS42. Lysis was successfully obtained following induction, killing the bacteria in about 40 minutes. This demonstrates that with an appropriate inducible promoter, in addition to exposing the immunogen gene or protein to the host's immune machinery, the cassette will cause lysis, providing assurance that the bacteria will not survive beyond their mission. Thus, in one embodiment, invasive reduced genome bacteria comprise a vector comprising an inducible lysis system that causes lysis of the bacteria upon induction.

Second, the protective effect of an exogenous restriction/modification system was demonstrated in MDS42. The pvuIIMR genes from Proteus vulgaris encode methylase and endonuclease functions. DNA that is not modified by specific methylation at the restriction sequences for the endonuclease is degraded. A plasmid encoding this system was transferred into MDS42. In a new host the methylase is expressed first and protects the host genome. Once the plasmid carrying the genes is established, the endonuclease is expressed and any DNA that subsequently enters the bacteria is degraded. Phage lambda was prepared in a wild type K-12 strain (no PvuII methylation) and then tested it on MDS42 with or without the restriction plasmid. Phage titers were at least three orders of magnitude lower on the restrictive host. This demonstrates that the protective effect of restriction against horizontal DNA transfer from the environment in the mammalian gut can be achieved. Defense against horizontal gene transfer is important as phage infection and plasmid transfer can bring drug resistance genes and virulence factors into a therapeutic strain if it is unprotected. Thus, in one embodiment, invasive reduced genome bacteria comprise a vector comprising an exogenous restriction/modification system.

### EXAMPLE 14

### Design of Stx2 Mutants via Epitope Scrambling

Synthetic genes were created encoding mosaic proteins consisting of multiple peptide epitopes of Shiga toxin 2 (Stx2) in scrambled order. DNA vaccines comprising these genes are expected to provide protection against a lethal challenge with the native toxin. For the vaccines, MDS bacteria (e.g. MDS42) expressing invasin will deliver either recombinant protein synthesized from a bacterial promoter during culture, or will deliver plasmid DNA encoding the synthetic genes at high copy number, preferably by the oral route. In the DNA vaccine, a eukaryotic promoter (e.g. CMV promoter) drives expression of the synthetic vaccine peptide once inside the target cell. In neither case is any purification of the immunogenic molecule necessary. Preparation of the vaccine would consist of bacterial fermentation then dilution of the culture to the dose concentration. Oral delivery of the vaccines would access the immune system by bactofection from the intestine.

To evaluate the concept, synthetic peptide vaccines were designed to provide protection against a lethal challenge with Shiga toxin 2 (Stx2). First, Stx2A (active site) subunit protein sequence (GenPept Accession No. AAZ73249) and Stx2B protein sequence (GenPept Accession No. AAZ73250) were scanned by a set of computer programs for regions of potential immunogenicity and prediction of B-cell epitopes.

The predicted B-cell epitopes were examined in the context of the entire Stx2A and Stx2B proteins and some were rejected that were unlikely to occur in the native mature toxin (in the signal sequence; across a cysteine bridge). Next, predicted peptide locations in the X-ray crystal structure of Stx2A and Stx2B were examined. This confirmed that the chosen epitopes were indeed exposed on the surface of the protein. Three Stx2A candidate peptides, StxA-1 (SEQ ID NO: 1), SNA-4 (SEQ ID NO: 2) and StxA-6 (SEQ ID NO: 3) and one Stx2B candidate peptide, StxB-1 (SEQ ID NO: 4) were synthesized and used to generate hybridomas. StxA-1 corresponds to amino acids 228-250 of Stx2A; StxA-4 corresponds to amino acids 61-75 of Stx2A; StxA-6 corresponds to amino acids 198-212 of Stx2A; and StxB-1 corresponds to amino acids 22-39 of Stx2B. Supernatants were screened to confirm monoclonal antibody (mAb) production, reactivity and specificity.

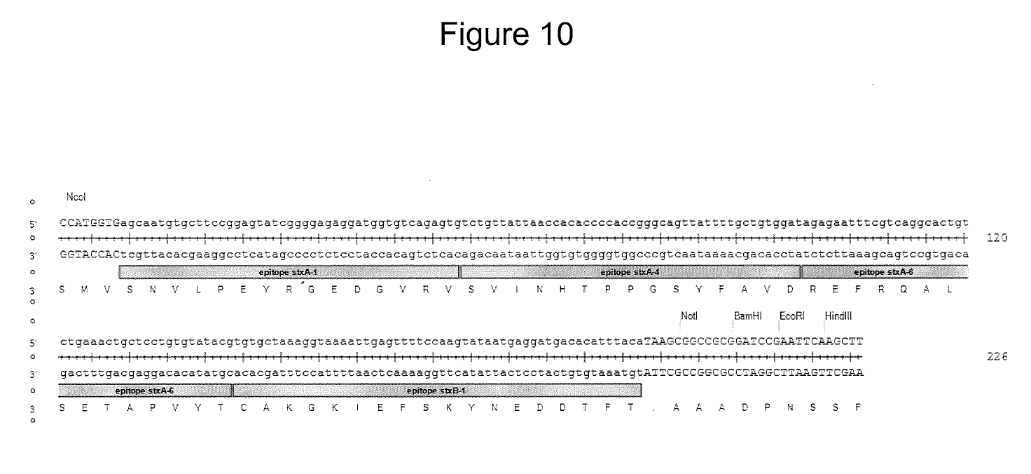

After immunogenicity of the peptides was confirmed, vaccine gene designs were made based on the peptide sequences of the epitopes. In one embodiment, the DNA sequences were codon-optimized for E. coli expression, and the peptides were simply combined end-to-end, in frame, though not in the order in which they occur in the Stx2 genes (SEQ ID NO: 5). See FIG. 10. The DNA sequence of this embodiment encodes a polypeptide comprising epitopes StxA-1, StxA-4, StxA-6 and StxB-1 without linker peptides separating the epitopes (SEQ ID NO: 6). Restriction sites were added to the sequence 5′ and 3′ of the gene for cloning into expression vectors. See FIG. 10.

Expression vectors carrying these genes will be used to transform reduced genome bacteria (e.g. MDS42) which will then be used to prepare doses for immunization of mice by IP injection and oral gavage. The ability of these vaccines to protect against a lethal challenge of Shiga toxin will be assessed.

Genes may be created encoding one or more Stx2 epitopes selected from the group consisting of SEQ ID NOs: 1-4 in any order. The genes may be created such that the gene is expressed as a single polypeptide comprising contiguous (i.e. end-to-end) Stx2 epitopes. Alternatively, the genes may be created such that short spacer (or linker) segments are added between the epitope-encoding sequences. In this embodiment, the gene is expressed as a single polypeptide comprising two or more Stx2 epitopes separated by spacer (or linker) peptides 1 to 20 residues in length. In other words, the linker peptides may be 1, 2, 3, 4, 5, 6, 7, 8, 9, 10, 11, 12, 13, 14, 15, 16, 17, 18, 19 or 20 residues in length. Linker peptides in single polypeptides comprising more than two Stx2 epitopes need not all be the same length.

Genes may be created such that the Stx2 epitopes are expressed in any order, including, without limitation: SEQ ID NOs: 1, 2, 3, 4; SEQ ID NOs: 1, 2, 4, 3; SEQ ID NOs: 1, 3, 2, 4; SEQ ID NOs: 1, 3, 4, 2; SEQ ID NOs: 1, 4, 2, 3; SEQ ID NOs: 1, 4, 3, 2; and so on. In each gene, the epitopes may be separated by spacer peptides.

TABLE 1
FIRST COMPLETED DELETIONS

| Deletion | Endpoints ^{a} | Size (bp) | Description ^{b} |
|---|---|---|---|
| MD1 | 263080, 324632 | 61553 | b0246-b0310; includes K-islands #16, 17, 18, CP4-6, eaeH |
| MD2 | 1398351, 1480278 | 81928 | b1336-b1411; includes K-island #83, Rac |
| MD3 | 2556711, 2563500 | 6790 | b2441-b2450; includes K-island #128, CP-Eut |
| MD4 | 2754180, 2789270 | 35091 | b2622-b2660; includes K-island #137, CP4-57, ileY |
| MD5 | 2064327, 2078613 | 14287 | b1994-b2008; includes K-islands # 94, 95, 96, CP4-44 |
| MD6 | 3451565, 3467490 | 15926 | b3323-b3338; includes K-islands #164, 165 |
| MD7 | 2464565, 2474198 | 9634 | b2349-b2363; includes K-island #121 |
| MD8 | 1625542, 1650785 | 25244 | b1539-b1579; includes K-island #77, Qin |
| MD9 | 4494243, 4547279 | 53037 | b4271-b4320; includes K-island #225, fec operon, fim operon |
| MD10 | 3108697, 3134392 | 25696 | b2968-b2987; includes K-island #153, glc operon |
| MD11 | 1196360, 1222299 | 25940 | b1137-b1172; includes K-island #71, e14 |
| MD12 | 564278, 585331 | 21054 | b0538-b0565; includes K-island #37, DLP12 |

TABLE 2
Transformation Efficiencies for E. coli Strains MDS31, MDS39 and
DH10B

| | DH10B | MDS31 | MDS39 |
|---|---|---|---|
| | (transformants per | (transformants per | (transformants per |
| | microgram DNA) | microgram DNA) | microgram DNA) |
| pBR322 | 2 × 10⁸ | 2.2 × 10⁸ | 2.7 × 10⁸ |
| Methylated | 2 × 10⁶ | 0.6 × 10⁶ | 1.2 × 10⁶ |
| BAC | | | |
| Unmethylated | 1.8 × 10⁶ | 4.0 × 10³ | 3.0 × 10⁶ |
| BAC | | | |

TABLE 3
Deleted Periplasmic Protein Genes

| Deletion | Gene, b# | MR Gene | MR Gene Product |
|---|---|---|---|
| GP16 | b1920 | fliY | cysteine transport protein (ABC superfamily, peri_bind) |
| GP16 | b1919 | yedO | D-cysteine desulfhydrase, PLP-dependent |
| GP2 | b0578 | nfnB | dihydropteridine reductase, o2-sensitive NAD(P)H reductase |
| GP4 | b0365 | tauA | taurine transport protein (ABC superfamily, peri_bind) |
| GP9 | b1329 | mppA | periplasmic murein tripeptide transport protein; negative |
| | | | regulator of antibacterial resistance |
| MD2 | b1386 | tynA | copper amine oxidase (tyramine oxidase) |
| MD6 | b3338 | chiA | endochitinase, periplasmic |
| MD9 | b4316 | fimC | periplasmic chaperone required for type 1 fimbrae |
| MD9 | b4290 | fecB | KpLE2 phage-like element; citrate dependent Fe(III) transport |
| | | | protein (ABC superfamily, peri_bind) |
| GP7 | b3047 | yqiH | putative periplasmic chaperone |
| MD1 | b0282 | yagP | putative periplasmic regulator |
| GP12 | b3215 | yhcA | putative periplasmic chaperone |

TABLE 4
Transformation Efficiencies for E. coli Strains MG1655,
MDS40 and DH10B

| | DH10B | MG1655 | MDS40 |
|---|---|---|---|
| | (transformants per | (transformants per | (transformants per |
| | microgram) | microgram) | microgram) |
| pUC19 | 1.3 × 10⁸ | 2.9 × 10⁸ | 1.3 × 10⁸ |
| BAC | 8.8 × 10⁶ | 3 × 10⁶ | 6.5 × 10⁶ |

TABLE 5
Transformation Efficiencies for E. coli Strains MG1655, MDS40 and
DH10B

| | DH10B | MG1655 | MDS40 |
|---|---|---|---|
| | (transformants per | (transformants per | (transformants per |
| | microgram) | microgram) | microgram) |
| pUC19 | 4.5 × 10⁵ | 3.7 × 10⁴ | 1.6 × 10⁴ |

TABLE 6

| | | Average | | |
|---|---|---|---|---|
| | | Doubling | | |
| Media | Strain | time | Std dev | Max OD |
| MOPS Minimal | MG1655 | 120.41 | 0.63 | 0.82 |
| MOPS Minimal | MDS12 | 123.43 | 6.91 | 0.61 |
| MOPS Minimal | MDS39 | 129.57 | 2.30 | 0.62 |
| MOPS Minimal | MDS40 | 128.26 | 5.30 | 0.61 |
| MOPS Minimal | DH10B | No growth | | |
| Rich Defined | MG1655 | 38.38 | 0.25 | 0.83 |
| Rich Defined | MDS12 | 49.05 | 4.05 | 0.84 |
| Rich Defined | MDS39 | 54.38 | 1.05 | 0.85 |
| Rich Defined | MDS40 | 51.19 | 1.77 | 0.86 |
| Rich Defined | DH10B | 45.40 | 2.30 | 0.62 |

TABLE 7

| MDS12 | MDS40 | MDS73 | del | lend | rend |
|---|---|---|---|---|---|
| deleted | deleted | deleted | MD1 | 263080 | 324632 |
| deleted | deleted | deleted | MD2 | 1398351 | 1480278 |
| deleted | deleted | deleted | MD3 | 2556711 | 2563500 |
| deleted | deleted | deleted | MD4 | 2754180 | 2789270 |
| deleted | deleted | deleted | MD5 | 2064327 | 2078613 |
| deleted | deleted | deleted | MD6 | 3451565 | 3467490 |
| deleted | deleted | deleted | MD7 | 2464565 | 2474198 |
| deleted | deleted | deleted | MD8 | 1625542 | 1650785 |
| deleted | deleted | deleted | MD9 | 4494243 | 4547279 |
| deleted | deleted | deleted | MD10 | 3108697 | 3134392 |
| deleted | deleted | deleted | MD11 | 1196360 | 1222299 |
| deleted | deleted | deleted | MD12 | 564278 | 585331 |
| | deleted | deleted | GP1 | 15388 | 20562 |
| | deleted | deleted | GP2 | 602688 | 608572 |
| | deleted | deleted | GP3 | 2507651 | 2515959 |
| | deleted | deleted | GP4 | 379334 | 387870 |
| | deleted | deleted | GP5 | 389122 | 399029 |
| | deleted | deleted | GP6 | 2993014 | 2996890 |
| | deleted | deleted | GP7 | 3182797 | 3189712 |
| | deleted | deleted | GP8 | 687083 | 688267 |
| | deleted | deleted | GP9 | 1386912 | 1396645 |
| | deleted | deleted | GP10 | 2099418 | 2135738 |
| | deleted | deleted | GP11 | 2284421 | 2288200 |
| | deleted | deleted | GP12 | 3359797 | 3365277 |
| | deleted | deleted | GP13 | 3648921 | 3651342 |
| | deleted | deleted | GP14 | 1128620 | 1140209 |
| | deleted | deleted | GP15 | 1960590 | 1977353 |
| | deleted | deleted | GP16 | 1995135 | 2021700 |
| | deleted | deleted | GP17 | 4553059 | 4594581 |
| | deleted | deleted | GP18 | 522062 | 529349 |
| | deleted | deleted | GP19 | 728588 | 738185 |
| | deleted | deleted | GP20 | 1525916 | 1531650 |
| | deleted | deleted | GP21 | 3616623 | 3623310 |
| | deleted | deleted | GP22 | 3759620 | 3767869 |
| | deleted | deleted | GP23 | 1041254 | 1049768 |
| | deleted | deleted | GP24 | 1085330 | 1096545 |
| | deleted | deleted | GP25 | 2163173 | 2175230 |
| | deleted | deleted | GP26 | 3578769 | 3582673 |
| | deleted | deleted | GP27 | 3718263 | 3719704 |
| | deleted | deleted | MD40 | 167484 | 173447 |
| | | deleted | GP28 | 331595 | 376535 |
| | | deleted | GP29 | 1588878 | 1599265 |
| | | deleted | GP30 | 3794575 | 3805725 |
| | | deleted | GP31 | 3886064 | 3904195 |
| | | deleted | GP32 | 2599182 | 2612802 |
| | | deleted | GP33 | 3738738 | 3752058 |
| | | deleted | GP34 | 4055987 | 4073034 |
| | | deleted | GP35 | 1349431 | 1364839 |
| | | deleted | GP36 | 2876592 | 2885242 |
| | | deleted | GP37 | 149715 | 156883 |
| | | deleted | GP38 | 674793 | 682616 |
| | | deleted | GP39 | 997082 | 1003880 |
| | | deleted | GP40 | 2318063 | 2334712 |
| | | deleted | gp41 | 3503000 | 3510000 |
| | | deleted | gp42 | 4304000 | 4311000 |
| | | deleted | gp43 | 557000 | 563000 |
| | | deleted | gp44 | 764000 | 770000 |
| | | deleted | gp45 | 1555000 | 1561000 |
| | | deleted | gp46 | 2382000 | 2388000 |
| | | deleted | gp47 | 2447000 | 2453000 |
| | | deleted | gp48 | 4547600 | 4553000 |
| | | deleted | gp50 | 747000 | 752000 |
| | | deleted | gp51 | 1727000 | 1732000 |
| | | deleted | gp52 | 2859000 | 2864000 |
| | | deleted | gp53 | 4488000 | 4493000 |
| | | deleted | gp54 | 2520000 | 2524000 |
| | | deleted | gp55 | 4086000 | 4090000 |
| | | deleted | gp56 | 1250000 | 1253000 |
| | | deleted | gp57 | 1650000 | 1653000 |
| | | deleted | gp58 | 2186000 | 2189000 |
| | | deleted | gp59 | 2474000 | 2477000 |
| | | deleted | gp60 | 3358000 | 3360000 |
| | | deleted | gp61 | 3864000 | 3866000 |

TABLE 8
genes (identified by b-number) deleted for each deletion strain

| | |
|---|---|
| MD1: | b0247, b0248, b0249, b0250, b0251, b0252, b0253, b0254, b0255, b0256, b0257, |
| | b0258, b0259, b0260, b0261, b0262, b0263, b0264, b0265, b0266, b0267, b0268, |
| | b0269, b0270, b0271, b0272, b0273, b0274, b0275, b0276, b0277, b0278, b0279, |
| | b0280, b0281, b0282, b0283, b0284, b0285, b0286, b0287, b0288, b0289, b0290, |
| | b0291, b0292, b0293, b0294, b0295, b0296, b0297, b0298, b0299, b0300, b0301, |
| | b0302, b0303, b0304, b0305, b0306, b0307, b0308, b0309, b0310 |
| MD2: | b1337, b1338, b1339, b1340, b1341, b1342, b1343, b1344, b1345, b1346, b1347, |
| | b1348, b1349, b1350, b1351, b1352, b1353, b1354, b1355, b1356, b1357, b1358, |
| | b1359, b1360, b1361, b1362, b1363, b1364, b1365, b1366, b1367, b1368, b1369, |
| | b1370, b1371, b1372, b1373, b1374, b1375, b1376, b1377, b1378, b1379, b1380, |
| | b1381, b1382, b1383, b1384, b1385, b1386, b1387, b1388, b1389, b1390, b1391, |
| | b1392, b1393, b1394, b1395, b1396, b1397, b1398, b1399, b1400, b1401, b1402, |
| | b1403, b1404, b1405, b1406, b1407, b1408, b1409, b1410, b1411 |
| MD3: | b2442, b2443, b2444, b2445, b2446, b2447, b2448, b2449, b2450 |
| MD4: | b2622, b2623, b2624, b2625, b2626, b2627, b2628, b2629, b2630, b2631, b2632, |
| | b2633, b2634, b2635, b2636, b2637, b2638, b2639, b2640, b2641, b2642, b2643, |
| | b2644, b2645, b2646, b2647, b2648, b2649, b2650, b2651, b2652, b2653, b2654, |
| | b2655, b2656, b2657, b2658, b2659, b2660 |
| MD5: | b1994, b1995, b1996, b1997, b1998, b1999, b2000, b2001, b2002, b2003, b2004, |
| | b2005, b2006, b2007, b2008 |
| MD6: | b3323, b3324, b3325, b3326, b3327, b3328, b3329, b3330, b3331, b3332, b3333, |
| | b3334, b3335, b3336, b3337, b3338 |
| MD7: | b2349, b2350, b2351, b2352, b2353, b2354, b2355, b2356, b2357, b2358, b2359, |
| | b2360, b2361, b2362, b2363 |
| MD8: | b1540, b1541, b1542, b1543, b1544, b1545, b1546, b1547, b1548, b1549, b1550, |
| | b1551, b1552, b1553, b1554, b1555, b1556, b1557, b1558, b1559, b1560, b1561, |
| | b1562, b1563, b1564, b1565, b1566, b1567, b1568, b1569, b1570, b1571, b1572, |
| | b1573, b1574, b1575, b1576, b1577, b1578, b1579 |
| MD9: | b4271, b4272, b4273, b4274, b4275, b4276, b4277, b4278, b4279, b4280, b4281, |
| | b4282, b4283, b4284, b4285, b4286, b4287, b4288, b4289, b4290, b4291, b4292, |
| | b4293, b4294, b4295, b4296, b4297, b4298, b4299, b4300, b4301, b4302, b4303, |
| | b4304, b4305, b4306, b4307, b4308, b4309, b4310, b4311, b4312, b4313, b4314, |
| | b4315, b4316, b4317, b4318, b4319, b4320 |
| MD10: | b2969, b2970, b2971, b2972, b2973, b2974, b2975, b2976, b2977, b2978, b2979, |
| | b2980, b2981, b2982, b2983, b2984, b2985, b2986, b2987 |
| MD11: | b1138, b1139, b1140, b1141, b1142, b1143, b1144, b1145, b1146, b1147, b1148, |
| | b1149, b1150, b1151, b1152, b1153, b1154, b1155, b1156, b1157, b1158, b1159, |
| | b1160, b1161, b1162, b1163, b1164, b1165, b1166, b1167, b1168, b1169, b1170, |
| | b1171, b1172 |
| MD12: | b0538, b0539, b0540, b0541, b0542, b0543, b0544, b0545, b0546, b0547, b0548, |
| | b0549, b0550, b0551, b0552, b0553, b0554, b0555, b0556, b0557, b0558, b0559, |
| | b0560, b0561, b0562, b0563, b0564, b0565 |
| GP1: | b0016, b0017, b0018, b0019, b0020, b0021, b0022 |
| GP2: | b0577, b0578, b0579, b0580, b0581, b0582 |
| GP3: | b2389, b2390, b2391, b2392, b2393, b2394, b2395 |
| GP4: | b0358, b0359, b0360, b0361, b0362, b0363, b0364, b0365, b0366, b0367, b0368 |
| GP5: | b0370, b0371, b0372, b0373, b0374, b0375, b0376, b0377, b0378, b0379, b0380 |
| GP6: | b2856, b2857, b2858, b2859, b2860, b2861, b2862, b2863 |
| GP7: | b3042, b3043, b3044, b3045, b3046, b3047, b3048 |
| GP8: | b0656 |
| GP9: | b1325, b1326, b1327, b1328, b1329, b1330, b1331, b1332, b1333 |
| GP10: | b2030, b2031, b2032, b2033, b2034, b2035, b2036, b2037, b2038, b2039, b2040, |
| | b2041, b2042, b2043, b2044, b2045, b2046, b2047, b2048, b2049, b2050, b2051, |
| | b2052, b2053, b2054, b2055, b2056, b2057, b2058, b2059, b2060, b2061, b2062 |
| GP11: | b2190, b2191, b2192 |
| GP12: | b3215, b3216, b3217, b3218, b3219 |
| GP13: | b3504, b3505 |
| GP14: | b1070, b1071, b1072, b1073, b1074, b1075, b1076, b1077, b1078, b1079, b1080, |
| | b1081, b1082, b1083 |
| GP15: | b1878, b1879, b1880, b1881, b1882, b1883, b1884, b1885, b1886, b1887, b1888, |
| | b1889, b1890, b1891, b1892, b1893, b1894 |
| GP16: | b1917, b1918, b1919, b1920, b1921, b1922, b1923, b1924, b1925, b1926, b1927, |
| | b1928, b1929, b1930, b1931, b1932, b1933, b1934, b1935, b1936, b1937, b1938, |
| | b1939, b1940, b1941, b1942, b1943, b1944, b1945, b1946, b1947, b1948, b1949, |
| | b1950 |
| GP17: | b4325, b4326, b4327, b4328, b4329, b4330, b4331, b4332, b4333, b4334, b4335, |
| | b4336, b4337, b4338, b4339, b4340, b4341, b4342, b4343, b4344, b4345, b4346, |
| | b4347, b4348, b4349, b4350, b4351, b4352, b4353, b4354, b4355, b4356, b4357, |
| | b4358 |
| GP18: | b0497, b0498, b0499, b0500, b0501, b0502 |
| GP19: | b0700, b0701, b0702, b0703, b0704, b0705, b0706 |
| GP20: | b1456, b1457, b1458, b1459, b1460, b1461, b1462 |
| GP21: | b3482, b3483, b3484 |
| GP22: | b3593, b3594, b3595, b3596 |
| GP23: | b0981, b0982, b0983, b0984, b0985, b0986, b0987, b0988 |
| GP24: | b1021, b1022, b1023, b1024, b1025, b1026, b1027, b1028, b1029, b1030, b1031 |
| GP25: | b2080, b2081, b2082, b2083, b2084, b2085, b2086, b2087, b2088, b2089, b2090, |
| | b2091, b2092, b2093, b2094, b2095, b2096 |
| GP26: | b3441, b3442, b3443, b3444, b3445, b3446 |
| GP27: | b3557, b3558 |
| MD40: | b0150, b0151, b0152, b0153 |
| GP28: | b0315, b0316, b0317, b0318, b0319, b0320, b0321, b0322, b0323, b0324, b0325, |
| | b0326, b0327, b0328, b0329, b0330, b0331, b0333, b0334, b0335, b0336, b0337, |
| | b0338, b0339, b0340, b0341, b0342, b0343, b0344, b0345, b0346, b0347, b0348, |
| | b0349, b0350, b0351, b0352, b0353, b0354 |
| GP29: | b1507, b1508, b1509, b1510, b1511, b1512 |
| GP30: | b3622, b3623, b3624, b3625, b3626, b3627, b3628, b3629, b3630, b3631, b3632 |
| GP31: | b3707, b3708, b3709, b3710, b3711, b3712, b3713, b3714, b3715, b3716, b3717, |
| | b3718, b3719, b3720, b3721, b3722, b3723 |
| GP32: | b2481, b2482, b2483, b2484, b2485, b2486, b2487, b2488, b2489, b2490, b2491, |
| | b2492 |
| GP33: | b3573, b3574, b3575, b3576, b3577, b3578, b3579, b3580, b3581, b3582, b3583, |
| | b3584, b3585, b3586, b3587 |
| GP34: | b3871, b3872, b3873, b3874, b3875, b3876, b3877, b3878, b3879, b3880, b3881, |
| | b3882, b3883, b3884 |
| GP35: | b1289, b1290, b1291, b1292, b1293, b1294, b1295, b1296, b1297, b1298, b1299, |
| | b1300, b1301, b1302 |
| GP36: | b2754, b2755, b2756, b2757, b2758, b2759, b2760, b2761 |
| GP37: | b0135, b0136, b0137, b0138, b0139, b0140, b0141 |
| GP38: | b0644, b0645, b0646, b0647, b0648, b0649, b0650 |
| GP39: | b0938,, b0939, b0940, b0941, b0942, b0943, b0944, b0945 |
| GP40: | b2219, b2220, b2221, b2222, b2223, b2224, b2225, b2226, b2227, b2228, b2229, |
| | b2230 |
| gp41: | b3376, b3377, b3378, b3379, b3380, b3381, b3382, b3383 |
| gp42: | b4084, b4085, b4086, b4087, b4088, b4089, b4090 |
| gp43: | b0530, b0531, b0532, b0533, b0534, b0535 |
| gp44: | b0730, b0731, b0732 |
| gp45: | b1483, b1484, b1485, b1486, b1487 |
| gp46: | b2270, b2271, b2272, b2273, b2274, b2275 |
| gp47: | b2332, b2333, b2334, b2335, b2336, b2337, b2338 |
| gp48: | b4321, b4322, b4323, b4324 |
| gp50: | b0716, b0717, b0718, b0719 |
| gp51: | b1653, b1654, b1655 |
| gp52: | b2735, b2736, b2737, b2738, b2739, b2740 |
| gp53: | b4265, b4266, b4267, b4268, b4269 |
| gp54: | b2405, b2405, b2407, b2408, b2409 |
| gp55: | b3897, b3898, b3899, b3900, b3901 |
| gp56: | b1201 |
| gp57: | b1580, b1581 |
| gp58: | b2108, b2109, b2110, b2111, b2112 |
| gp59: | b2364, b2365 |
| gp60: | b3213, b3214 |
| gp61: | b3686, b3687, b3688, b3689, b3690 |

TABLE 9

| | | | SCBal./M9 | | | | SCBal./M9-BirA | | | |
|---|---|---|---|---|---|---|---|---|---|---|
| | | | 155 | | 156 | | 157 | | 158 | |
| Virus | Clade | R5/X4 | ID50 | ID90 | ID50 | ID90 | ID50 | ID90 | ID50 | ID90 |
| | | | Format 1 | | | | | | | |
| IIIB | B | TCLA-X4 | 26 | 4 | 37 | 5 | 32 | 4 | 15 | 1 |
| 2005 | B | X4 | 211 | 22 | 507 | 25 | 69 | 7 | 101 | 10 |
| 2044 | B | X4 | 255 | 28 | 317 | 54 | 96 | 5 | 82 | 9 |
| 89.6 | B | R5/X4 | 72 | 9 | 116 | 12 | 47 | 5 | 17 | 2 |
| ADA | B | R5 | 52 | 7 | 68 | 9 | 34 | 4 | 23 | 2 |
| SI05 | B | R5 | 137 | 18 | 206 | 28 | 35 | 4 | 65 | 6 |
| SF162 | B | R5 | 94 | 12 | 219 | 31 | 45 | 6 | 35 | 4 |
| Bal. | B | R5 | 78 | 9 | 226 | 34 | 72 | 8 | 38 | 3 |
| 92UG024 | D | X4 | 88 | 10 | 176 | 19 | 162 | 23 | 201 | 28 |
| 93BR020 | F | R5/X4 | 808 | 136 | 1045 | 115 | 89 | 10 | 506 | 10 |
| 92UG021 | D | X4 | 153 | 22 | 285 | 41 | 105 | 12 | 88 | 7 |
| | | | Format 2 | | | | | | | |
| 2044 | B | X4 | 67 | 8 | 58 | 8 | 297 | 34 | 28 | 3 |
| 2075 | B | R5/X4 | 75 | 10 | 74 | 10 | 525 | 92 | 243 | 48 |
| 93BR020 | F | R5/X4 | 37 | 4 | 109 | 18 | 356 | 47 | 841 | 42 |
| 92UG021 | D | X4 | 42 | 5 | 64 | 7 | 333 | 38 | 1241 | 31 |
| SI07 | B | R5/X4 | 46 | 4 | 43 | 6 | 258 | 31 | 388 | 33 |

1. A method for introducing and expressing one or more nucleic acids or genes in an animal cell comprising:
(a) providing a vector comprising a low-copy number origin of replication, an inducible high copy number origin of replication, and one or more eukaryotic expression cassettes, said expression cassettes comprising said one or more nucleic acids or genes;
(b) transforming at least one invasive reduced genome Escherichia coli bacterium with the vector to form at least one transformed bacterium; and
(c) infecting the animal cell with said transformed bacterium.

2. (canceled)

3. The method of claim 1, wherein the low-copy number origin of replication is oriS.

4. (canceled)

5. The method of claim 1, wherein the high-copy number origin of replication is oriV.

6. The method of claim 5, wherein the high-copy number origin of replication is regulated by a polypeptide encoded by a gene under the control of an arabinose promoter.

7. The method of claim 6, wherein said polypeptide is a TrfA.

8. The method of claim 1, wherein said transformed bacterium is frozen in an aqueous glycerol solution prior to said infecting.

9. The method of claim 8, wherein said aqueous glycerol solution is 15% w/w glycerol.

10. The method of claim 8, wherein said transformed bacterium is frozen to a temperature of about −80° C.

11. (canceled)

12. (canceled)

13. The method of claim 1, wherein the Escherichia coli strain is MD42.

14. (canceled)

15. (canceled)

16. The method of claim 1, wherein said invasive ability of the bacterium is conferred by one or more Yersinia genes.

17. The method of claim 1, wherein the animal cell is a human cell.

18. The method of claim 1 wherein
said one or more expression cassettes comprise at least a gene encoding the transcription factor Oct3/4 and a gene encoding a member of the SRY-related HMG-box (Sox) transcription factor family, wherein said animal cell is a mammalian somatic cell and wherein expression of said transcription factors causes the generation of an iPS cell from the mammalian somatic cell.

19. The method of claim 18, wherein the member of the Sox transcription factor family is Sox2.

20. The method of claim 18, wherein the one or more eukaryotic expression cassettes further comprises a gene encoding a transcription factor selected from the group consisting of: Nanog, Lin28, Klf1, KlG₅ Klf4 and Klf5.

21. The method of claim 20, wherein the one or more eukaryotic expression cassettes further comprises a gene encoding Klf4.

22-36. (canceled)

37. The method of claim 18, where the mammalian somatic cell is a human fibroblast cell selected from the group consisting of: IMR90 fetal fibroblasts, postnatal foreskin fibroblasts, and adult dermal fibroblasts.

38. The method of claim 18, wherein the iPS cell possesses telomerase activity.

39. The method of claim 18, wherein the iPS cell expresses at least one selected marker selected from the group consisting of one or more of the following: SSEA-I(−), SSEA-3(+), SSEA-4(+), TRA-1-60(+), TRA-1-81(+) and TRA-2-49/6E.

40-46. (canceled)

47. A reduced genome bacterium prepared by the method of claim 1.

48. The bacterium of claim 47, wherein said nucleic acid or gene is under the control of a cardiac-specific promoter.

49. The bacterium of claim 48, wherein the cardiac specific promoter is selected from: an α-myosin heavy chain promoter; a β-myosin heavy chain promoter; a myosin light chain-2v promoter; a myosin light chain-2a promoter; cardiomyocyte-restricted cardiac ankyrin repeat (CARP) promoter; cardiac α-actin promoter; ANP promoter; BNP promoter; cardiac troponin C promoter; cardiac troponin T promoter; and skeletal α-actin promoter.

50. The bacterium of claim 48 wherein said nucleic acid or gene is selected from: vascular endothelial growth factor (VEGF) 1; VEGF 2; fibroblast growth factor (FGF) 4; endothelial nitric oxide synthase (eNOS); heme oxygenase-1 (HO-I); extracellular superoxide dismutase (Ec-SOD); heat shock protein 70 (HSP70); Bcl-2; hypoxia-inducible factor 1 (HIF-I) alpha; sarcoplasmic reticulum Ca²⁺ ATPase (SERCA); sarcoplasmic reticulum Ca²⁺-adenosinetriphosphatase-2 (SERCA2); and sulfonylurea receptor-2 (SUR2).

51. The method of claim 1, wherein the animal cell is a cardiomyocyte.

52. The method of claim 51, wherein the cardiomyocyte is a human cardiomyocyte.

53. The method of claim 52, wherein the gene or nucleic acid is under the control of a cardiac-specific promoter.

54. The method of claim 53, wherein the cardiac specific promoter is selected from: vascular endothelial growth factor (VEGF) 1; VEGF 2; fibroblast growth factor (FGF) 4; endothelial nitric oxide synthase (eNOS); heme oxygenase-1 (HO-I); extracellular superoxide dismutase (Ec-SOD); heat shock protein 70 (HSP70); Bcl-2; hypoxia-inducible factor 1 (HIF-I) alpha; sarcoplasmic reticulum Ca²⁺ ATPase (SERCA); sarcoplasmic reticulum Ca²⁺-adenosinetriphosphatase-2 (SERCA2); and sulfonylurea receptor-2 (SUR2).

55. The method of claim 1, wherein the animal cell is a stem cell.

56. The method of claim 55, wherein the stem cell is a hematopoietic, mesenchymal or cardiac stem cell.

57-58. (canceled)

59. An isolated nucleic acid according to claim 60 comprising a sequence selected from the group consisting of:
(a) the sequence set forth as SEQ ID NO: 5;
(b) nucleotides 9-197 of SEQ ID NO: 5;
(c) a sequence at least 90% identical to any one of (a)-(b); and
(d) a sequence at least 95% identical to any one of (a)-(b).

60. An isolated nucleic acid comprising a sequence encoding a polypeptide comprising two or more amino acid sequences selected from the group consisting of:
(a) the sequence set forth as SEQ ID NO: 1;
(b) the sequence set forth as SEQ ID NO: 2;
(c) the sequence set forth as SEQ ID NO: 3;
(d) the sequence set forth as SEQ ID NO: 4; and
(e) a sequence at least 90% identical to any one of (a)-(d),
wherein said two or more amino acid sequences are separated by a linker peptide of from 0 to 20 amino acids in length.

61. An isolated nucleic acid according to claim 60 comprising a sequence encoding a polypeptide comprising the sequence of SEQ ID NO: 6.

62. (canceled)

63. An expression vector comprising a nucleic acid according to claim 60 operably linked to a promoter.

64. A method for introducing and expressing the nucleic acid according to claim 60 in an animal cell comprising:
(a) providing a vector comprising a first origin of replication, a second origin of replication, and a eukaryotic expression cassette, said expression cassette comprising said nucleic acid;
(b) transforming at least one invasive reduced genome bacterium with the vector to form at least one transformed bacterium;
(c) freezing said transformed bacterium in an aqueous glycerol solution; and
(d) infecting the animal cell with said transformed bacterium.

65. A reduced genome bacterium prepared by the method of claim 64.

66. A polypeptide encoded by a nucleic acid of claim 60


SEQUENCE LISTING (6 sequences)

SEQ ID NO: 1 - protein, 15 aa, Artificial Sequence
  Stx2A epitope 1
SNVLPEYRGEDGVRV

SEQ ID NO: 2 - protein, 15 aa, Artificial Sequence
  Stx2A epitope 2
SVINHTPPGSYFAVD

SEQ ID NO: 3 - protein, 15 aa, Artificial Sequence
  Stx2A epitope 3
REFRQALSETAPVYT

SEQ ID NO: 4 - protein, 18 aa, Artificial Sequence
  Stx2B Epitope 1
CAKGKIEFSKYNEDDTFT

SEQ ID NO: 5 - DNA, 226 bp, Artificial Sequence
  Synthetic DNA encoding scrambled Stx2 epitopes
ccatggtgagcaatgtgcttccggagtatcggggagaggatggtgtcagagtgtctgttattaaccacaccccaccgggcagttattttgctgtggatagagaatttcgtcaggcactgtctgaaactgctcctgtgtatacgtgtgctaaaggtaaaattgagttttccaagtataatgaggatgacacatttacataagcggccgcggatccgaattcaagctt

SEQ ID NO: 6 - protein, 63 aa, Artificial Sequence
  Synthetic polypeptide comprising scrambled Stx2 epitopes end to end
SNVLPEYRGEDGVRVSVINHTPPGSYFAVDREFRQALSETAPVYTCAKGKIEFSKYNEDDTFT